(19) United States
(12) Patent Application Publication
(10) Pub. No.: US 2006/0199179 A1
(43) Pub. Date: Sep. 7, 2006

(54) Method for diagnosis of colorectal tumors

(75) Inventors: Yusuke Nakamura, Yokohama-shi (JP); Yoichi Furukawa, Kawasaki-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Kanagawa (JP); The University of Tokyo, Tokyo (JP)

(21) Appl. No.: 10/518,938

(22) PCT Filed: Dec. 5, 2002
(86) PCT No.: PCT/JP02/12760

§ 371 Date: Apr. 5, 2006

Related U.S. Application Data
(60) Provisional application No. 60/389,994, filed on Jun. 19, 2002.

(51) Int. Cl. C12Q 1/68; C12P 19/34

(52) U.S. Cl. 435/6; 435/91.2

(57) ABSTRACT

The invention provides objective methods for detecting and diagnosing colorectal cancers and pre-malignant lesions. For example, the methods disclosed herein can reliably detect very early-stage colorectal cancers. In one embodiment, the diagnostic method involves the scoring of gene expression profiles that discriminate between adenomas and carcinomas. The profile score calculated acts as diagnostic indicator that can objectively indicate whether a sample tissue is non-cancerous, pre-cancerous, or cancerous. The present invention further provides methods of diagnosing colorectal tumors in a subject, methods of screening for therapeutic agents useful in the treatment of colorectal tumors, methods of treating colorectal tumors and method of vaccinating a subject against colorectal tumors.

### TECHNICAL FIELD

The present invention relates to the field of cancer research. More particularly, the present invention relates to methods for detecting colorectal cancer and objectively distinguishing between colorectal adenomas and carcinomas. The invention further relates to methods of diagnosing colorectal tumors in a subject, methods of screening for therapeutic agents useful in the treatment of colorectal tumors, methods of treating colorectal tumors and method of vaccinating a subject against colorectal tumors.

### BACKGROUND OF THE INVENTION

The invention relates to detection and diagnosis of tumors, particularly colorectal tumors.

Colorectal carcinoma is a leading cause of cancer deaths in developed countries. Specifically, more than 130,000 new cases of colorectal cancer in the United States are reported each year. Colorectal cancer represents about 15% of all cancers. Of these, approximately 5% are directly related to inherited genetic defects. Many patients have a diagnosis of pre-cancerous colon or rectal polyps before the onset of cancer. While many small colorectal polyps are benign, some types may progress to cancer.

The most widely used screening test for colorectal cancer is colonoscopy. This method is used to visualize a suspicious growth and/or take a tissue biopsy. Typically, the tissue biopsy is histologically examined and a diagnosis delivered based on the microscopic appearance of the biopsied cells. However, this method is limited in that it yields subjective results and can not be used for very early detection of pre-cancerous states. The development of a sensitive, specific and convenient diagnostic system for detecting very early-stage colorectal cancers or pre-malignant lesions is highly desirable as it could ultimately eliminate this disease.

The present invention represents a marked improvement in the field of colon cancer detection and diagnosis. Prior to the invention, knowledge of genes involved in colorectal tumors was fragmentary. The information described herein provides genome-wide information about how gene expression profiles are altered during multi-step carcinogenesis. Specifically, the present invention describes genes that discrimiate between colorectal adenomas and carcinomas, referred to herein as “marker genes”. On the basis of expression of selected “marker” genes, a scoring system was established that can assist clinicians in distinguishing adenomas from carcinomas. The information disclosed herein not only contributes to a more profound understanding of colorectal tumorigenesis, particularly of adenoma-carcinoma progression, but also provide indicators for developing novel strategies to diagnose, treat, and ultimately prevent colorectal carcinomas.

### SUMMARY OF THE INVENTION

Accordingly, the present invention provides diagnostic methods that correlate the expression of marker genes to the presence or absence of colorectal cancer. More particularly, the present invention provides sensitive, specific and convenient diagnostic methods for distinguishing between malignant and pre-malignant lesions and diagnosing the presence of colorectal cancer in a subject. For example, the diagnostic methods of the present invention can reliably detect very early-stage colorectal cancers.

The marker genes of the present invention are characterized as being either up regulated or down-regulated in colorectal tumors. Up-regulated marker genes include, RNA/protein processing genes, oncogenes (e.g., HMGIY, DEK and NPM1), cell adhesion/cytoskeleton molecules (e.g., TUBB, K-ALPHA, TGFBI, CDH3 and PAP), growth control molecules (e.g., IMPDH2 and ODC1), signal transduction molecules (e.g., BRF1, PLAB, LAP18, CD81 and MICMARCKS), cell-cycle control molecules (e.g., RAN and UBE2I), transcription factors (e.g., HMG1 and HMG2), as well as tumor-associated molecules such as PPP2R1B, LDBB and SLC29A1. Marker genes commonly up-regulated in colorectal tumors are set forth in Table 1. Marker genes were up-regulated in colorectal adenoma as compared to normal tissues, and no significant difference in marker gene expression was observed between carcinoma and normal tissue (Table 3). Marker genes were up-regulated in colorectal carcinoma as compared to normal tissues, and no significant difference in marker gene expression was observed between adenoma and normal tissue (Table 4).

Colorectal tumor-associated down-regulated marker genes include associated with programmed cell death (e.g. CASP8, CASP9, CFLAR, DFFA, PAWR TNF, TNFRSF10C and TNFRSF12. Further down-regulated marker genes include, immune modulators (e.g., chemokine receptors such as IL1RL2, IL17R and IL3RA), growth suppression molecules (e.g., Suppressin, DCN, MADH2 and SS, tumor suppression molecules (e.g., TP53), cell adhesion/cytoskeleton molecules (e.g, ADAM8, AVIL, CDH17, CEACAM1, CTNNA2, ICAPA, KRT9, and ARHGAP5), metabolic factors (e.g., BPH-L, CA2, CASA, HSD11B2 and ECHS1), ion transporters (e.g., SLC15A2, SLC22A1, SLC4A3 and SLC5A1), a natural antimicrobial molecule (e.g. DEFA6). Marker genes commonly down-regulated in colorectal tumors are set forth in Table 2.

In the present invention, the term “colorectal tumor” refers to both colorectal adenoma and colorectal carcinoma Marker genes listed in Table 3 and Table 4 are useful as stage specific markers of colorectal adenoma and colorectal carcinoma, respectively. On the other hand, marker genes listed in Table 1 and Table 2 are general marker genes for colorectal tumors. The term “general marker” employed herein means that the existence of that marker proves the existence of some tumor including adenoma and carcinoma.

In the diagnostic methods of the present invention, it is preferable that multiple marker genes are selected for comparison of expression levels thereof. The more marker genes selected for comparison, the more reliable the diagnosis. The expression levels of a number of genes can be compared conveniently by using an expression profile. The term “expression profile” refers to a collection of expression levels of a number of genes, preferably marker genes that are differentially expressed in colorectal carcinoma as compared to colorectal adenoma.

Accordingly, in one embodiment, the present invention provides a method for diagnosing colorectal tumors in a subject comprising the steps of:
(a) detecting an expression level of one or more marker genes in a specimen collected from a subject to be diagnosed, wherein the one or more marker genes is selected from the group consisting of the genes listed in Table 1 and the genes listed in Table 2; and
(b) comparing the expression level of the one or more marker genes to that of a control, wherein high expression level of a marker gene from Table 1 or a low expression level of a marker gene from Table 2, as compared to control, is indicative of colorectal cancer.

The expression levels of marker genes in a particular specimen can be estimated by quantifying mRNA corresponding to, or protein encoded by, the marker genes. Quantification methods for mRNA are known to those skilled in the art. For example, the levels of mRNAs corresponding to the marker genes can be estimated by Northern blotting or RT-PCR. Since all the nucleotide sequences of the marker genes are known, anyone skilled in the art can design nucleotide sequences of probes or primers to quantify the marker genes.

Also the expression level of the marker genes can be analyzed based on the activity or amount of proteins encoded by the marker genes. A method for determining the amount of marker proteins is shown below. For example, immunoasssays are useful to detect/quantify the protein in a biological material. Any biological material can be used for the detection/quantification of the protein or it's activity. For example, a blood sample is analyzed to determine the protein encoded by serum marker. Alternatively, a suitable method can be selected to determine the activity of proteins encoded by the marker genes according to the activity of each protein analyzed.

Expression levels of the marker genes in a specimen (test sample) are estimated and compared with those in a normal sample. When such a comparison shows that the expression level of a marker gene set forth in Table 1 is higher than that in the normal sample, the subject is judged to be affected with a colorectal tumor. The expression level of marker genes in specimens from a normal individual and a subject may be determined at the same time. Alternatively, normal ranges of the expression levels can be determined by a statistical method based on the results obtained by analyzing the expression level of the marker genes in specimens previously collected from a control group. A result obtained by examining the sample of a subject is compared with the normal range and when the result does not fall within the normal range, the subject is judged to be affected with a colorectal tumor. Similarly, colorectal adenoma and / or carcinoma may be diagnosed using marker genes set forth in Table 3 or Table 4, respectively.

In the present invention, a diagnostic agent for diagnosing colorectal tumor, adenoma, and/or carcinoma is also provided. The diagnostic agent of the present invention comprises a compound that binds to the DNA or protein of a marker gene. Preferably, an oligonucleotide that hybridizes to the polynucleotide of a marker gene, or an antibody that specifically binds to the protein encoded by a marker gene may be used as the compound.

The present invention further provides a method for diagnosing colorectal cancer in a subject comprising the step of comparing the marker gene expression profile of a sample specimen collected from a subject with the marker gene expression profile of a control (i.e. a non-cancerous) specimen. When expression profiling analysis shows that the expression profile contains characteristics of colorectal cancer, the subject is judged to be affected with the disease. Specifically, when not all but most of the marker genes exhibit colorectal cancer-associated patterns of alterations of gene expression levels, the expression profile comprising those of the marker genes has characteristics of colorectal cancer. For example, when 50% or more, preferably 60% or more, more preferably 80% or more, still more preferably 90% or more of the marker genes constituting the expression profile exhibit colorectal cancer-associated patterns of alterations in gene expression levels, one can safely conclude that the expression profile has characteristics of colorectal cancer.

In a preferred embodiment, the marker genes comprise genes up-regulated in colorectal carcinomas as compared with colorectal adenomas, such as those shown in Table 4. Alternatively, the marker genes may comprise genes unregulated in colorectal adenomas as compared with colorectal carcinomas, such as those shown in Table 3. Multiple marker genes from various categories may also be selected. Specifically, the present invention provides a method of identifying adenoma comprising the steps of:
(a) detecting an expression level of one or more marker genes in a specimen collected from a subject to be diagnosed, wherein the one or more marker genes is selected from the group consisting of the genes listed in Table 3; and
(b) comparing the expression level of the one or more marker genes to that of a control, wherein high expression level of a marker gene from Table 3 as compared to control is indicative of adenoma.

Furthermore, the present invention provides a method of identifying carcinoma comprising the steps of:
(a) detecting an expression level of one or more marker genes in a specimen collected from a subject to be diagnosed, wherein the one or more marker genes is selected from the group consisting of the genes listed in Table 4; and
(b) comparing the expression level of the one or more marker genes to that of a control, wherein high expression level of a marker gene from Table 4 as compared to control is indicative of carcinoma

Clinically important information can be obtained by distinguishing between adenoma and carcinoma Adenoma is a pre-cancerous tumor, whereas carcinoma is a cancerous tumor requiring treatment. Any of the marker genes listed in Tables 3 and 4 are used in the present method for identifying carcinoma Alternatively, expression levels of one or more marker gene selected from Table 3 and one or more marker gene selected from Table 4 may be detected for the identification of carcinoma according to the present invention. Compared to an identification using one or more marker gene from either Table 3 or 4, a more accurate identification can be achieved by confirming elevated expression of one or more marker gene selected from Table 3 and no significant changes in the expression of one or more marker gene from Table 4, or elevated expression of one or more marker gene selected from Table 4 and no significant changes in the expression of one or more marker gene from Table 3.

In an alternate embodiment, the diagnostic method of the present invention involves the step of scoring expression profiles for genes that discriminate between adenomas and carcinomas. The steps of the method include receiving expression profiles for genes selected as differentially expressed in adenomas versus carcinomas (i.e., “marker genes”) and determining a function of the log ratios of the expression profiles over the selected genes. The step of “determining a function of the log ratios of the expression profiles over the selected genes” may comprise summing the weighted log ratios of the expression profiles over the selected genes. The weight for each gene is assigned a first value when the average log ratio is higher for carcinomas than for adenomas and a second value when the average log ratio is lower for carcinomas than for adenomas. Preferably, the second value is substantially the opposite of the first value, e.g., the first value is 1 and the second value is −1.

The method of the present invention further provides a diagnostic determination of the cancer status of a tissue sample. For example, in one embodiment, the diagnostic method of the present invention preferably involves the steps of measuring the level of expression of a gene in a test sample, e.g., a tumor biopsy or a biopsy of a normal tissue, and determining a gene expression ratio value for each of a plurality of differentially-expressed index (or marker) genes. The gene expression ratio corresponds to the amount of expression in the test sample as compared to the amount of expression in normal tissue. A sign [e.g., a plus sign (+) or a minus sign (−)] is assigned for each value. The sign is +1 if ave_{carcinoma} is greater than ave_{adenoma} and said sign is −1 is ave_{carcinoma} is less than ave_{adeonoma}.

Each value is combined to determine a diagnostic indicator, which objectively indicates whether a tissue is pre-cancerous, or cancerous. For example, the indicator discriminates between adenomas and carcinomas.

In another embodiment, the method includes the step of determining a ratio of expression for each of a plurality of selected marker genes in the tissue and combining indicia of the ratios to determine a cancer value. The combining of a particular ratio for a particular gene influences the cancer value toward a carcinogenic indication if the particular gene is associated with (indicative of) carcinoma (i.e., a carcinoma marker gene) and influences the cancer value toward an adenoma indication if the particular gene is associated with (indicative of) at least one of adenoma and normal (i.e., an adenoma marker gene). Preferably, the plurality is greater than 10 genes, more preferably greater than 25 genes, more preferably greater than 40 genes, and most preferably greater than 50 genes.

A significant advantage of the diagnostic methods of the present invention is that the diagnostic determination is made objectively rather than subjectively. Earlier methods were limited because they relied on the subjective examination of histological samples. Another advantage of the diagnostic methods of the present invention is sensitivity. The methods described herein can discriminate among normal, pre-cancerous, and cancerous tissue very early in the carcinogenic process, whereas subjective histological examination cannot be used for very early detection of pre-cancerous states.

The present invention further provides methods for treating colorectal tumors, such as colorectal adenomas and colorectal carcinomas. The present invention revealed that expression levels of certain discriminating marker genes are significantly increased (i.e., up-regulation) or decreased (i.e., down-regulation) in colorectal tumors as compared to normal epithelia (see genes listed Tables 1 and 2) and/or in colorectal carcinomas as compared to colorectal adenomas (see genes listed in Table 3 and 4). Accordingly, any of these marker genes can be used as a target in treating the colorectal tumors. Specifically, when the expression level of a marker gene is elevated in a colorectal tumor (up-regulation; e.g., genes of Table1, 3, and 4), then the condition can be treated by reducing expression levels or suppressing its activities. Methods for controlling the expression levels of marker genes are known to those skilled in the art. For example, an antisense nucleic acids or RNAi (RNA interference) corresponding to the nucleotide sequence of the marker gene can be administered to reduce the expression level of the marker gene. Alternatively, an antibody against the protein encoded by the marker gene can be administered to inhibit the biological activity of the protein.

Conversely, when the expression level of a marker gene is decreased in colorectal tumors (down regulation; e.g., genes of Table 2), then the condition can be treated by increasing the expression level or enhancing the activity. For example, colorectal tumors can be treated by administering a protein encoded by a down-regulated marker gene. The protein may be directly administered to the patient or, alternatively, may be expressed in vivo subsequent to being introduced into the patient, for example, by administering an expression vector or host cell carrying the down-regulated marker gene of interest. Suitable mechanisms for in vivo expression of a gene of interest are known in the art. Alternatively, colorectal tumors can be treated by administering an antibody that binds to a protein encoded by an up-regulated marker gene of interest. In a further embodiment, colorectal tumors can be treated by administering an antisense nucleic acids against an up-regulated marker gene of interest.

In addition to providing methods of treating colorectal tumors, the invention also provides methods of preventing colorectal tumors, more particularly the onset and progression of colorectal cancer. Specifically, the present invention provides a method for vaccinating a subject against colorectal tumors comprising the step of administering a DNA corresponding to one or more marker genes, proteins encoded by a marker gene, or an antigenic fragment of such a protein, wherein the marker genes comprises a gene up-regulated in colorectal tumors, such as those listed in Table 1, Table 3, and Table 4. The vaccine may comprise multiple vaccine antigens corresponding to multiple up-regulated marker genes.

Marker genes listed in Tables 3 and 4 are specific marker genes of adenoma and carcinoma, respectively. However, in fact, malignant tumors are formed due to the progress of adenoma to carcinoma Thus, colorectal carcinoma can be prevented by preventing the onset of adenoma.

In a further embodiment, the present invention provides methods for screening candidate agents which are potential targets in the treatment of colorectal tumors. As discussed in detail above, by controlling the expression levels or activities of marker genes, one can control the onset and progression of colorectal cancer. Thus, candidate agents, which are potential targets in the treatment of colorectal tumors, can be identified through screenings that use the expression levels and activities of marker genes as indices. In the context of the present invention, such screening may comprise, for example, the following steps:
(1) contacting a candidate compound with a cell expressing one or more marker genes, wherein the one or more marker genes is selected from the group consisting of the genes listed in Table 1, Table 2, Table 3, and Table 4; and
(2) selecting a compound that reduces the expression level of one or more marker genes selected from Table 1, Table 3, and Table 4 as compared to a control or enhances the expression of one or more marker genes selected from Table 2 as compared to a control.
   Cells expressing a marker gene include, for example, cell lines established from colorectal cancer lesions; such cells can be used for the above screening of the present invention.

Alternatively, the screening method of the present invention may comprise the following steps:
(1) contacting a candidate compound with a protein encoded by a marker gene, wherein the marker gene is selected from the group consisting of the genes listed in Table 1, Table 2, Table 3, and Table 4;
(2) measuring the activity of said protein; and
(3) selecting a compound that reduces the activity of said protein when said marker gene is selected from Table 1, Table 3, and Table 4 or that enhances the activity of said protein when said marker gene is selected from Table 2.
   A protein required for the screening can be obtained as a recombinant protein using the nucleotide sequence of the marker gene. Based on the information of the marker gene, one skilled in the art can select any biological activity of the protein as an index for screening and a measurement method based on the selected biological activity.

Alternatively, the screening method of the present invention may comprise the following steps:
(1) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of the genes listed in Table 1, Table 2, Table 3, and Table 4;
(2) measuring the activity of said reporter gene; and
(3) selecting a compound that reduces the expression level of said reporter gene when said marker gene is selected from Table 1, Table 3, and Table 4 or that enhances the expression level of said reporter gene when said marker gene is selected from Table 2, as compared to a control.
   Suitable reporter genes and host cells are well known in the art. The reporter construct required for the screening can be prepared by using the transcriptional regulatory region of a marker gene. When the transcriptional regulatory region of a marker gene has been known to those skilled in the art, a reporter construct can be prepared by using the previous sequence information. When the transcriptional regulatory region of a marker gene remains unidentified, a nucleotide segment containing the transcriptional regulatory region can be isolated from a genome library based on the nucleotide sequence information of the marker gene.

Alternatively, the screening method of the present invention may comprise the following steps:
(1) administering a candidate compound to a test animal;
(2) measuring the expression level of one or more marker genes in a biological sample from the test animal, wherein the one or more marker genes is selected from the group consisting of the genes listed in Table 1, Table 2, Table 3, and Table 4;
(3) selecting a compound that reduces the expression level of one or more marker genes selected from Table 1, Table 3, and Table 4 as compared to a control or enhances the expression of one or more marker genes selected from Table 2 as compared to a control.

In the screening methods of the present invention wherein the expression level of the selected marker gene is decreased in colorectal tumors (i.e., down-regulated marker genes), compounds that have the activity to increase, compared to the control, the expression level of the gene should be selected as the candidate agents. Conversely, when a marker gene whose expression level is increased in colorectal tumors (i.e., unregulated marker genes) is selected in the screening method, compounds that have the activity of decreasing the expression level compared to the control should be selected as the candidate agents.

The marker genes listed in Tables 3 and 4 are specific marker genes of adenoma and carcinoma, respectively. However, in fact, malignant tumors are formed due to the advance of adenoma to carcinoma Thus, colorectal carcinoma can be prevented by preventing the onset of adenoma.

There is no limitation on the type of candidate compound used in the screening of the present invention. The candidate compounds of the present invention can be obtained using any of the numerous approaches of combinatorial library methods known in the art, including: biological library methods; spatially addressable parallel solid phase or solution phase library methods; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad Sci. USA 90:6909; Erb et al. (1994) Proc. Natl. Acad. Sci. USA 91:11422; Zuckermann et al. (1994). J. Med Chem. 37:2678; Cho et al. (1993) Science 261:1303; Carrell et al. (1994) Angew. Chem. Int. Ed Engl. 33:2059; Carell et al. (1994) Angew. Chem. Int. Ed. Engl. 33:2061; and Gallop et al. (1994) J. Med Chem. 37:1233. Libraries of compounds may be presented in solution (e.g., Houghten (1992) Bio Techniques 13:412), or on beads (Lam (1991) Nature 354:82), chips (Fodor (1993) Nature 364:555), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865) or phage (Scott and Smith (1990) Science 249:386; Devlin (1990) Science 249:404; Cwirla et al. (1990) Proc. Natl. Acad Sci. USA 87:6378; and Felici (1991) J. Mol. Biol. 222:301).(United States Published Patent Application 2002/0103360).

Other features and advantages of the invention will be apparent from the following detailed description and from the claims.

### BRIEF DESCRIPTION OF THE DRAWINGS

FIGS. 1A-B are diagrams of a two-dimensional hierarchical clustering of 771 genes across 20 colorectal tumors. The color in each well represents relative expression of each gene (vertical axis) in each paired sample (horizontal axis); more intense colors reflect wider differences between tumor and normal epithelium. Red, increased in tumor; green, decreased; black, unchanged; gray, no expression in the tumor cells. In the sample axis, carcinomas (IT) and adenomas (P) were separated to two different trunks. In the gene axis, 771 genes were clustered in different branches according to their similarity; the shorter the branches the greater the similarity. Sub-clusters A and B were selected for further analysis.

FIG. 2 is a diagram representing fifty-one genes, the expression of which was found to be up-regulated in both adenomas and carcinomas. (Cy3/Cy5)ave indicates average value of Cy3/Cy5 in the 20 paired samples. Genes that appear repeatedly represent the same genes spotted on different set of slides. Red, increased in tumor, green, decreased; black, unchanged; gray, no expression in the tumor cells.

FIGS. 3A-B are diagrams representing the functional clusters in the gene axis. FIG. 3A shows ten of 24 genes in cluster A (genes whose expression is more abundant in carcinomas than in adenomas), and FIG. 3B shows 12 of 29 genes in cluster B (genes whose expression is more abundant in adenomas than in carcinomas). Bold italic type indicates genes that are related to bioenergetics homeostasis. Genes that appear repeatedly represent the same genes spotted on different set of slides.

FIGS. 4A-B are bar graphs showing a validation of microarray data. FIG. 4A shows Log2(Cy3/Cy5) values of 20 samples (11 carcinomas and 9 adenomas) in cDNA microarray analysis. FIG. 4B shows Log2(Tumor/Normal) values for 13 additional samples (6 carcinomas and 7 adenomas) obtained by QPCR. The expression ratio (Tumor: Normal) of TGFB1, LAP13, HECH, NME1, TCEA1 and PSMA7 determined by QPCR were in line with the microarray data for all six genes. Data are presented here as 10-90th percentiles of calculated values. Statistical significance was examined by Mann-Whitney U tests.

FIG. 5A is a diagram representing clustering analysis. The data for each gene were first median-centered, and an “Average Linkage Clustering” was subsequently applied to the data set (red, data>median value; green, data<median value). In the sample axis, 25 tumors were separated to two trunrs (adenoma group and carcinoma group). Asterisks (*) indicate additional test samples. A sample, 056P3, was diagnosed as an early adenocarcinoma by histological examination. In the gene axis, the 18 genes on the top showed higher expression in carcinoma than in adenoma, and were labeled with “1” as a sign. The 32 genes at the bottom showed higher expression in adenoma than in carcinoma, and were labeled with “−1” as a sign. Statistical significance was examined by the Mann-Whitney U test.

FIG. 5B is a diagram representing Molecular Diagnosis Scores (MDSs). The data were presented as 10-90th percentiles of the calculated values. Asterisks denote the five additional samples for validating the MDS system. Tumor 056P3 is a well-differentiated adenocarcinoma.

### DETAILED DESCRIPTION

In the context of the present invention, the following definitions apply:

Tumors of the colorectal epithelium are classified as benign, malignant or pre-malignant. In the context of the present invention, the term “colorectal tumors” encompasses benign, malignant and pre-malignant tumors of the epithelium of the colon or rectal. The term “colorectal cancer” refers to a malignant state, characterized by uncontrolled, abnormal growth of cells. Cancer cells can spread locally or though the blood stream and lymphatic system to other parts of the body.

A “carcinoma” is a malignant new growth of cells that arises from the epithelium. Carcinomas are cancerous tumors that tend to infiltrate into adjacent tissue and metastasize to distant organs. An adenocarcinoma is a specific type of carcinoma arising from the lining of the walls of an organ, such as colon or rectum. Herein, the terms “carcinoma” and “adenocarcinoma” are used interchangeably. There is a clear need in the art for new methods for diagnosing, treating and preventing colorectal carcinoma, particularly at the early stages—before to the carcinoma metastasizes to other organ systems.

An “adenoma” is a benign epithelial tumor in which the cells form a recognizable glandular structure or in which the cells are clearly derived from glandular epithelium. Many colon cancers have been demonstrated to develop through the “adenoma-to-carcinoma sequence” model in the literature (auto et al., (1975) Cancer, 36, 2251-2270). Accordingly, in colorectal tumors, adenoma is the pre-malignant phase of colorectal carcinoma Early detection and diagnosis of adenoma is useful in preventing the onset of carcinormalikewise, the treatment and prevention of adenoma can protect the progressing into colorectal carcinoma in a subject.

The present invention describes genes that discriminate between colorectal tumors and normal epithelium as well as genes that discriminate between adenomas and carcinomas. Such genes are herein collectively referred to as “marker genes”. The present invention demonstrates that the expression of such marker genes can be analyzed to distinguish between tumor cells from normal cells, more preferably adenomas (i.e., benign or pre-malignant tumors) and carcinomas (i.e., malignant tumors).

The term “expression profile” as used herein refers to a collection of expression levels of a number of genes. In the context of the present invention, the expression profile preferably comprises marker genes that discriminate between adenomas and carcinomas. The present invention involves the step of analyzing expression profiles of marker genes to determine if a sample displays characteristics of colorectal cancer, thereby distinguishing colorectal cancers from pre-malignant lesions and diagnosing the presence of colorectal cancer in a subject.

The term “characteristics of a colorectal cancer” is used herein to refer to a pattern of alterations in the expression levels of a set of marker genes which is characteristic to colorectal cancer. Specifically, certain marker genes are described herein either up-regulated or down-regulated in colorectal cancer. When the expression level of one or more up-regulated marker genes included in the expression profile is elevated as compared with that in a control, the expression profile can be assessed as having the characteristics of colorectal cancer. Likewise, when the expression level of one or more down-regulated marker genes included in the expression profile is lowered as compared with that of a control, the expression profile can be assessed as having the characteristics of colorectal cancer. When, not all, but most of the pattern of alteration in the expression levels constituting the expression profile is characteristic to colorectal cancer, the expression profile is assessed to have the characteristics of colorectal cancer.

In the context of the present invention, expression profiles can be obtained by using a “DNA array”. A “DNA array” is a device that is convenient for comparing expression levels of a number of genes at the same time. DNA array -based expression profiling can be carried out, for example, by the method as disclosed in “Microarray Biochip Technology” (Mark Schena, Eaton Publishing, 2000), etc.

A DNA array comprises immobilized high-density probes to detect a number of genes. In the present invention, any type of polynucleotide can be used as probes for the DNA array. Preferably, cDNAs, PCR products, and oligonucleotides are usefuil as probes. Thus, expression levels of many genes can be estimated at the same time by a single-round analysis. Namely, the expression profile of a specimen can be determined with a DNA array. The DNA array-based method of the present invention comprises the following steps of:

(1) synthesizing aRNAs or eDNAs including those of marker genes;

(2) hybridizing the aRNAs or cDNAs with probes for the marker genes; and

(3) detecting the aRNA or cDNA hybridizing with the probes and quantifyig the amount of mRNA thereof.

The term “aRNA” refers to RNA transcribed from a template cDNA with RNA polymerase (amplified RNA). A aRNA transcription kit for DNA array-based expression profiling is commercially available. With such a kit, aRNA can be synthesized using T7 promoter-attached CDNA as a template with T7 RNA polymerase. Alternatively, by PCR using random primer, CDNA can be amplified using, as a template, a CDNA synthesized from mRNA.

The DNA array may further comprise probes, which have been spotted thereon, to detect the marker genes of the present invention. There is no limitation on the number of marker genes spotted on the DNA array. For example, one may select 5% or more, preferably 20% or more, more preferably 50% or more, still more preferably 70 % or more of the marker genes of the present invention. Genes other than the marker genes may be also spotted on the DNA array. For example, a probe for a gene whose expression level is not significantly altered may be spotted on the DNA array. Such a gene can be used for normalizing assay results to compare assay results of multiple arrays or different assays.

A “probe” is designed for each selected marker gene, and spotted on a DNA array. Such a “probe” may be, for example, an oligonucleotide comprising 5-50 nucleotide residues. A method for synthesizing such oligonucleotides on a DNA array is known to those skilled in the art. Longer DNAs can be synthesized by PCR or chemically. A method for spotting long DNA, which is synthesized by PCR or the like, onto a glass slide is also known to those skilled in the art. A DNA array that is obtained by the method as described above can be used for diagnosing colorectal cancer according to the present invention.

The prepared DNA array is contacted with aRNA, followed by the detection of hybridization between the probe and aRNA. The aRNA can be previously labeled with a fluorescent dye. A fluorescent dye such as Cy3(red) and Cy5 (blue) can be used to label a aRNA. aRNA s from subject and control are labeled with different fluorescent dyes, respectively. The difference in the expression level between the two can be estimated based on a difference in the signal intensity. The signal of fluorescent dye on the DNA array can be detected by a scanner and analyzed using a special program. For example, the Suite from Affymetrix is a software package for DNA array analysis.

The compound isolated by the screening is a candidate for drugs that inhibit the activity of the protein encoded by marker genes and can be applied to the treatment or prevention of colorectal tumors.

Moreover, compound in which a part of the structure of the compound inhibiting the activity of proteins encoded by marker genes is converted by addition, deletion and/or replacement are also included in the compounds obtainable by the screening method of the present invention.

When administrating the compound isolated by the method of the invention as a pharmaceutical for humans and other mammals, such as mice, rats, guinea-pigs, rabbits, chicken, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; and flavoring agents such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannnose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80 (TM) and HCO-50.

Sesame oil or Soy-bean oil can be used as a oleaginous liquid and may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol andphenol; and an anti-oxidant. The prepared injection may be filled into a suitable ampule.

Methods well known to one skilled in the art may be used to administer the pharmaceutical composition of the present inevntion to patients, for example as intraarterial, intravenous, or percutaneous injections and also as intranasal, transbronchial, intramuscular or oral administrations. The dosage and method of administration vary according to the body-weight and age of a patient and the administration method; however, one skilled in the art can routinely select a suitable metod of administration. If said compound is encodable by a DNA, the DNA can be inserted into a vector for gene therapy and the vector administered to a patient to perform the therapy. The dosage and method of administration vary according to the body-weight, age, and symptoms of the patient but one skilled in the art can suitably select them.

For example, although the dose of a compound that binds to the protein of the present invention and regulates its activity depends on the symptoms, the dose is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight

As noted above, antisense nucleic acids corresponding to the nucleotide sequence of a marker gene can be used to reduce the expression level of the marker gene. Antisense nucleic acids corresponding to marker genes that are up-regulated in colorectal carcinoma are useful for the treatment of colorectal carcinoma Specifically, the antisense nucleic acids of the present invention may act by binding to the marker genes or mRNAs corresponding thereto, thereby inhibiting the transcription or translation of the genes, promoting the degradation of the mRNAs, and/or inhibiting the expression of proteins encoded by the marker genes, finally inhibiting the function of the proteins . The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at 80% or higher, more preferably 90% or higher, even more preferably 95% or higher over a span of at least 15 continuous nucleotides. Algorithms known in the art can be used to determine the homology.

The antisense nucleic acid derivatives of the present invention act on cells producing the proteins encoded by marker genes by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid derivative of the present invention can be made into an external preparation, such as a liniment or a poultice, by mixing with a suitable base material which is inactive against the derivative.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative is given to the patient by directly applying onto the ailing site or by injecting into a blood vessel so that it will reach the site of ailment. An antisense-mounting medium can also be used to increase durability and membrane-permeability. Examples are, liposomes, poly-L-lysine, lipids, cholesterol, lipofectin or derivatives of these.

The dosage of the antisense nucleic acid derivative of the presentinvention can be adjusted suitably according to the patient's condition and used in desired amounts. For example, a dose range of 0.1 to 100 mg/kg, preferably 0.1 to 50 mg/kg can be administered.

The antisense nucleic acids of the invention inhibit the expression of the protein of the invention and is thereby useful for suppressing the biological activity of a protein of the invention. Also, expression-inhibitors, comprising the antisense nucleic acids of the invention, are useful since they can inhibit the biological activity of a protein of the invention.

The antisense nucleic acids of present invention include modified oligonucleotides. For example, thioated nucleotides may be used to confer nuclease resistance to an oligonucleotide.

The present invention refers to the use of antibodies, particularly antibodies against a protein encoded by an up-regulated marker gene, or a fragment of the antibody. As used herein, the term “antibody” refers to an immunoglobulin molecule having a specific structure, that interacts (i.e., binds) only with the antigen that was used for synthesizing the antibody (i.e., the up-regulated marker gene product) or with an antigen closely related to it. Furthermore, an antibody may be a fragment of an antibody or a modified antibody, so long as it binds to one or more of the proteins encoded by the marker genes. For instance, the antibody fragment may be Fab, F(ab′)2, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. These modification methods are conventional in the field.

Alternatively, an antibody may be obtained as a chimeric antibody, between a variable region derived from a nonhuman antibody and a constant region derived from a human antibody, or as a humanized antibody, comprising the complementarity determining region (CDR) derived from a nonhuman antibody, the frame work region (FR) derived from a human antibody, and the constant region. Such antibodies can be prepared by using known technologies.

The present invention provides preventative vaccines. In the context of the present invention, the term “vaccine” refers to antigenic formulations that induce immunity against colorectal tumors. The immunity may be transient and one or more booster administrations may be required.

The antigen within the vaccine may comprise a DNA corresponding to one or more up-regulated marker gene, such as those set forth in Table 1, or a protein encoded by such a marker gene or an antigenic fragment thereof. In the context of the present invention, the term “antigenic fragment” refers to a portion of a molecule, when introduced into the body, stimulates the production of an antibody specific to the marker gene of interest.

### Experimental Section

Prior to the present invention, knowledge of genes involved in colorectal tumors was fragmentary. Herein, expression profiles of pre-malignant and malignant lesions of the colon were examined and compared to provide information about genes that undergo altered expression during progression from adenoma to carcinoma. The data described herein provides genome-wide information about how expression profiles are altered during multi-step carcinogenesis.

To elucidate the mechanisms underlying the pathway from adenoma to carcinoma, gene-expression profiles of 20 colorectal tumors (9 adenomas and 11 differentiated adenocarcinomas) were analyzed by means of a CDNA microarray representing 23,040 genes coupled with laser-capture microdissection. Index genes (genes whose expression is different in carcinoma compared to adenoma or normal tissue) were identified. Specifically, 51 genes whose expression was consistently up-regulated and 376 that were consistently down-regulated in both types of tumors as compared to normal colonic epithelium were identified. Fifty. (50) genes whose expression levels were significantly different between adenomas and carcinomas were also identified. A two-dimensional hierarchical clustering analysis of expression profiles of the 20 tumors correctly separated the carcinoma group from the adenoma group. On the basis of expression profiles of the 50 discriminating genes, a scoring system was established to separate adenomas from carcinomas. Application of this scoring system to the evaluation of five additional colorectal tumors correctly predicted their cancer status, which was also independently determined by histological examination.

The scoring system of the present invention provides objective diagnostic information to assist clinicians in diagnosing colorectal tumors and distinguishing adenomas from carcinomas. The data reported herein provides valuable information to enhance understanding of colorectal carcinogenesis, to facilitate development of novel diagnostic strategies, and to identify molecular targets for therapeutic drugs and preventive agents.

The results of the “Molecular Diagnosis Score” (MDS) system using expression profiles of the 50 genes corroborated its feasibility for predicting the histological features of colorectal tumors. Analysis of gene-expression profiles of very early colon cancers is used to define a more precise cut-off value to distinguish between benign and malignant lesions. Nevertheless, since histological diagnosis of adenomas and carcinomas is sometimes very difficult and may vary among pathologists (Schlemper et al., 2000), the MDS system may ultimately be useful in distinguishing benign from malignant tumors because it enables an objective quantification of each tumor based on a genome-wide database.

Tissue samples from non-cancer, pre-cancerous, and cancerous tissues were obtained and analyzed as follows.

Tissue Samples and Laser-Capture Microdissection (LCM)

Eleven differentiated adenocarcinomas, 9 adenomas, and their corresponding normal mucosae of the colon were obtained from 16 patients who underwent colectomy. In four cases, both adenomas and carcinomas had arisen in the same patient. All 20-paired samples were embedded in TissueTek OCT medium (Miles, Inc.) and frozen at −80° C. Procedures of fixation, staining and LCM were performed using known methods, e.g., the method of Kitahara et al., 2001, Cancer Res ., 61, 3544-3549. About 10,000 cells were selectively collected by LCM from each tissue sample.

RNA Extraction, T7-Based RNA Amplification and cDNA Microarray

Extraction of total RNA and T7-based RNA amplification were carried out by standard methods. Two rounds of amplification yielded 15-80 μg of amplified RNA (aRNA) from each sample. A 2.5-μg aliquot of aRNA from each tumor and normal epithelium were labeled with Cy3-dCTP and Cy5-dCTP, respectively (Amersham Pharmacia Biotech). To reduce experimental fluctuation, duplicate sets of cDNA microarray slides containing 23,040 cDNAs for each analysis were used. Fabrication of the CDNA microarray slides, hybridization, washing, and detection of signals were carried out using methods known in the art. The 23,040 genes surveyed were selected from UniGene database (National Center for Biotechnology Information), and their cDNA fragments were amplified by RT-PCR using gene-specific primers for each gene and a variety of human polyA RNAs as template (Clontech).

Data Analysis

The intensity of each signal of Cy3 and Cy5 was evaluated photometrically using Array Vision software (Imaging Research Inc., St. Catherines, Ont. Canada) and normalized according to the expression of 52 housekeeping genes described by Kitahara et al, 2001, Cancer Res ., 61, 3544-3549. After normalization, each gene was separated into one of four categories based on the average Cy3/Cy5 ratio (r): up-regulated (r>2), down-regulated (r<0.5), unchanged (0.5<r<2) and low (expression level below cutoff level for detection). Excel, Cluster and TreeView software packages were used for subsequent analysis.

Validation of Data

To assess the reproducibility of hierarchical clustering, clustering results were compared in the sample axis by using different sets of genes. Specifically, 23,040 target sequences were spotted on five slides and clustering analysis was performed for the 20 samples in all five sets. When one sample consistently fell into the same cluster in different sets of genes, the data was defined as reproducible. The reproducibility was more than 80% when Cy3 or Cy5 fluorescent units were above 100,000. An average was calculated for Cy3- and Cy5-fluorescence intensities of each gene in all 20 cases. Genes were excluded from further analysis when both intensities fell below a cut-off of 1×10⁵ units. Accordingly, a total of 2,425 genes was selected.

Thus, 771 genes were chosen based on the criteria that the values were obtained in more than 16 cases (80%) and the standard deviations of observed values were grater than 5 0.5.

Calculation of “Molecular Diagnosis Score” (MS)

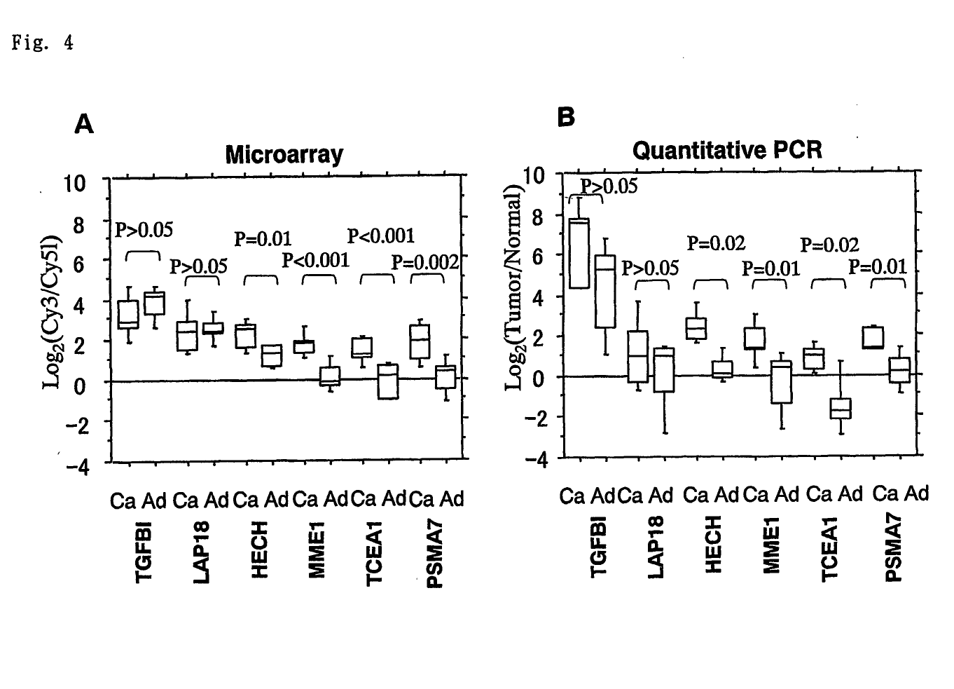

The MDS of each tumor was defined as the sum of weighted log ratios of expression profiles of the 50 genes selected as differentially expressed in adenomas vs. carcinomas: MDS₁=ΣASₖ log₂(rᵢₖ), where rᵢₖ is the expression ratio (Cy3/Cy5) of gene k of patient i, and Sₖ is the sign for gene k which was determined as follows. The first calculation was the determination of the average log ratio log₂(rᵢₖ) for gene k in the 11 adenocarcinomas and the 9 adenomas (ave_{carcinoma}=Σlog₂(rᵢₖ)/n_{carcinoma} and ave_{adenoma}=Σlog₂(rᵢₖ)/n_{adenoma}). Then, a sign (±) was determined for each gene: Sₖ=+1, if ave_{carcinoma}>ave_{adenoma}, and Sₖ=−1, if ave_{carcinoma}<ave_{adenoma} (FIG. 4A).

Real-Time Quantitative RT-PCR

To verify the microarray data, six genes were selected and their expression levels examined in 13 additional samples (7 adenomas and 6 carcinomas) by means of real-time quantitative RT-PCR (TaqMan PCR, Perkin-Elmer), using a 7700 Sequence Detector (Perkin-Elmer). Each single-stranded cDNA was reverse-transcribed from amplified RNA and diluted for subsequent PCR amplification. Malate dehydrogenase 1 (MDH1) served as a relative quantitative control since it showed the smallest Cy3/Cy5 fluctuations in 100 hybridizations. Each PCR was carried out in a 25-μl volume and amplified for 10 min at 95° C. for activation of AmpliTaq Gold™, followed by 40 cycles of 95° C. for 15 s and 60° C. for 1 min. The genes and sequences of the primers and probes used for quantitative RT-PCR are listed in Table A below.

TABLE A
Genes, Sequences of Primers and Probes used for quantitative RT-PCR

| | | |
|---|---|---|
| Symbol | Primer | Probe |

| | | |
|---|---|---|
| MDH1 | F:5′-TCCCTGTTGTAATCAAGAATAAGACCT-3′ | 5′-Vic-TTGTTGAAGGTCTCCCTATTA- |
| | R:5′-CAGTTCCTTTGCAGTAAGATCCATC-3′ | ATGATTTCTCACGTG-Tamra-3′ |
| TGFBI | F:5′-GCAGACTCTGCGCTTGAGATC-3′ | 5′-Fam-AACAAGCATCAGCGTTTTCC- |
| | R:5′-GGGCTAGTCGCACAGACCTC-3′ | AGGGCT-Tamra-3′ |
| LAP18 | F:5′-CAAATGGCTGCCAAACTGG-3′ | 5′-Fam-CGTTTGCGAGAGAAGGATAA |
| | R:5′-GGATTCTTTGTTCTTCCGCACT-3′ | GCACATTGAAG-Tamra-3′ |
| HECH | F:5′-GACAGCAGTGGAGAATTGATGTTT-3′ | 5′-Fam-TGAGGCAGACTTGGTGCTGG |
| | R:5′-ACAATTTGAGGACACTTCATATTTGC-3′ | CG-Tamra-3′ |
| NME1 | F:5′-GCATACAAGTTGGCAGGAACATTA-3′ | 5′-Fam-CATGGCAGTGATTCTGTGGA |
| | R:5′-ACCACAAGCCGATCTCCTTCT-3′ | GAGTGCA-Tamra-3′. |
| TCEA1 | F:5′-AGATGCGGAAAAACTTGACCA-3′ | 5′-Fam-AAGCCATCAGAGAGCATCAG |
| | R:5′-AGTCAGTCTGGGTCCCACCA-3′ | ATGGCC-Tamra-3′ |
| PSMA7 | F:5′-GCAGCGTTATACGCAGAGCA-3′ | 5′-Fam-TGGGCGCAGGCCGTTTGG- |
| | R:5′-ACCCACGATGAGGGCAGAG-3′ | Tamra-3′ |

Statistics

Assessment of statistical differences of gene expression in carcinomas vs. adenomas was determined by Mann-Whitney U tests. A P value ≦0.05 was considered statistically significant. Statistical analyses were performed using Stat View software.

Two-Dimensional Hierarchical Clustering

To analyze correlation among the samples and genes, a two-dimensional hierarchical clustering algorithm (http://www.microarrays.org/software) was applied to the gene expression data obtained from 20 tumors. Genes were excluded from further analysis when average Cy3- and Cy5-fluorescence intensities fell below 1×10⁵ units. This resulted in the selection of a set of genes whose values were obtained in more than 16 cases (80%).

Next, genes with standard deviations of observed values less than 0.5 were excluded. A total of 771 genes passed through this filter for subsequent clustering analysis.

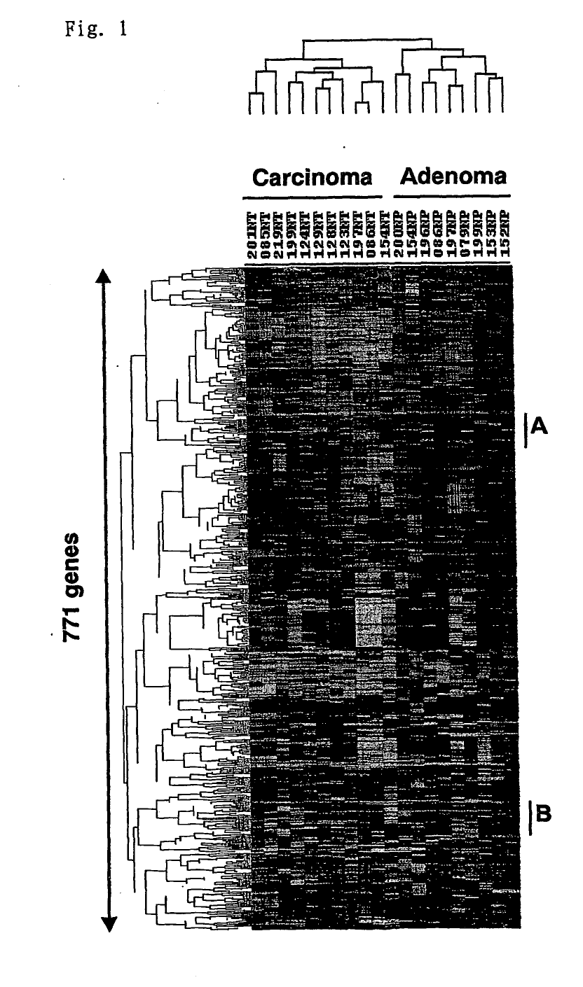

In the sample axis, the 20 samples were separated into two major groups based on their expression profiles; all of the 9 tumors belonging to one group were adenomas and the other major group consisted of the eleven carcinomas (FIGS. 1A-B). This result is consistent with a recent report that four colon adenomas were separated from 18 adenocarcinomas using oligonucleotide arrays (Notterman et al., 2001). The expression profiles obtained on the microarray clearly demonstrated that adenomas and adenocarcinomas have specific expression profiles,.and indicated that molecular classification of colonic tumors is feasible.

Up-Regulated Genes

Since many colon cancers arise from adenomas, genes involved in early stages of colorectal tumorigenesis are deregulated (compared to normal tissue) in both types of tumors. To identify such genes, genes were selected from a data set of 2,425 genes according to the following criteria: if the Cy3/Cy5 ratio of the gene was >2 in more than 50% of the tumors, it was defined as a consistently up-regulated gene, and if the ratio was <0.5 in more than 50% of the tumors, it was defined as consistently down-regulated.

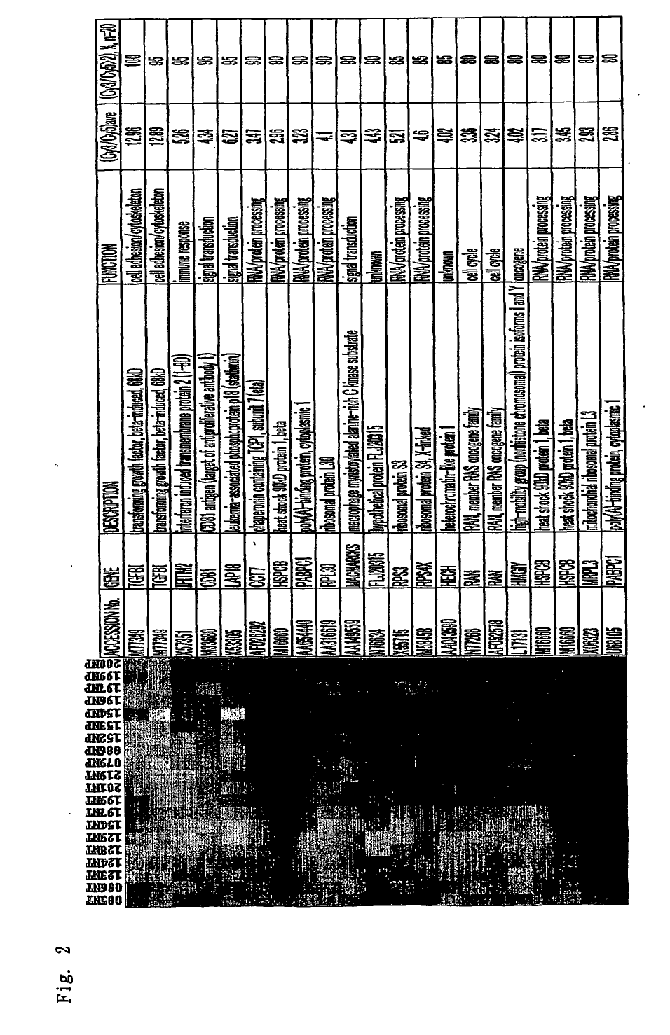

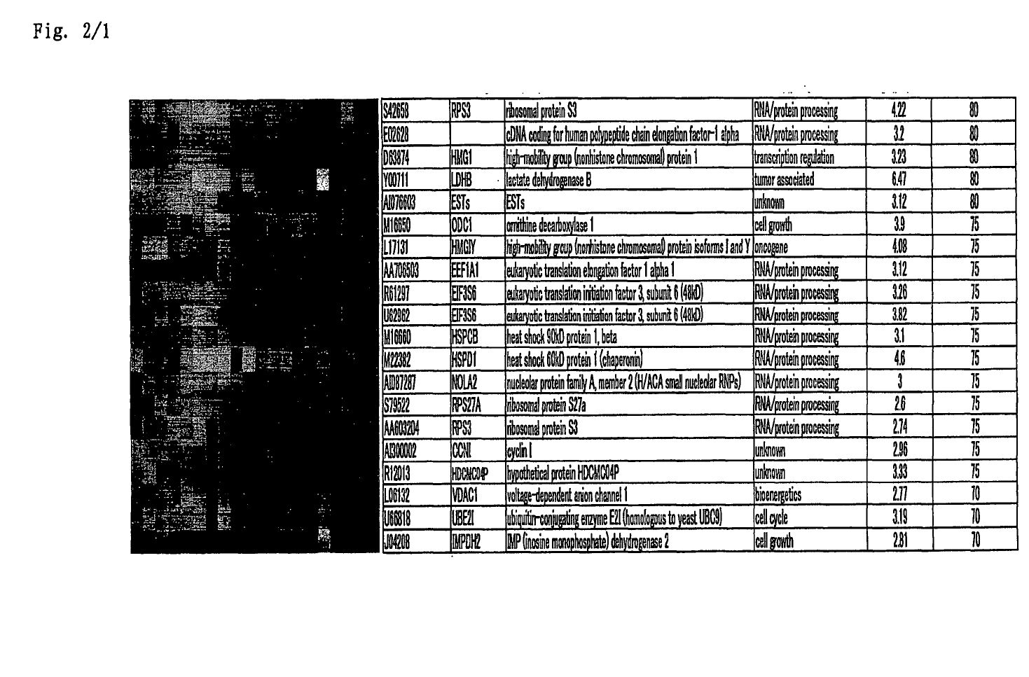

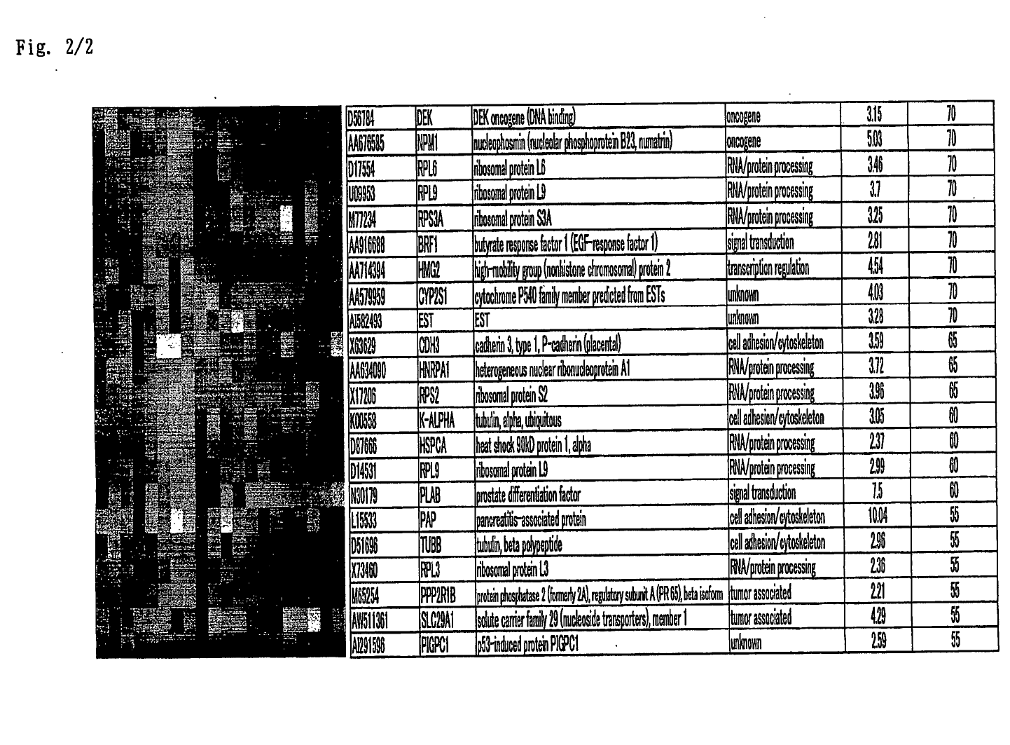

With these criteria, 51 genes were identified as commonly up-regulated in both tumor phenotypes as compared with their corresponding normal epithelia (FIG. 2). These commonly up-regulated genes are set forth in Table 1 below:

TABLE 1
Genes commonly up-regulated in colonic tumors

| | | | | | (Cy3/ |
|---|---|---|---|---|---|
| | ACCESSION | | | (Cy3/ | Cy5 < 0.5), |
| No. | No. | GENE | DESCRIPTION | Cy5)ave | %, n = 20 |
| 1 | M77349 | TGFBI | transforming growth factor, beta- | 12.96 | 100 |
| | | | induced, 68 kD | | |
| 2 | X57351 | IFITM2 | interferon induced transmembrane | 5.26 | 95 |
| | | | protein 2 (1-8D) | | |
| 3 | M33680 | CD81 | CD81 antigen (target of | 4.34 | 95 |
| | | | antiproliferative antibody 1) | | |
| 4 | X53305 | LAP18 | leukemia-associated | 6.27 | 95 |
| | | | phosphoprotein p18 (stathmin) | | |
| 5 | AF026292 | CCT7 | chaperonin containing TCP1, | 3.47 | 90 |
| | | | subunit 7 (eta) | | |
| 6 | M16660 | HSPCB | heat shock 90 kD protein 1, beta | 2.96 | 90 |
| 7 | AA654440 | PABPC1 | poly(A)-binding protein, | 3.23 | 90 |
| | | | cytoplasmic 1 | | |
| 8 | AA316619 | PRL30 | ribosomal protein L30 | 4.1 | 90 |
| 9 | AA149559 | MACMARCKS | macrophage myristoylated alanine- | 4.31 | 90 |
| | | | rich C kinase substrate | | |
| 10 | N76634 | FLJ20315 | hypothetical protein FLJ20315 | 4.43 | 90 |
| 11 | X55715 | RPS3 | ribosomal protein S3 | 5.21 | 85 |
| 12 | M58458 | RPS4X | ribosomal protein S4, X-linked | 4.6 | 85 |
| 13 | AA043590 | HECH | heterochromatin-like protein 1 | 4.02 | 85 |
| 14 | N77266 | RAN | RAN, member RAS oncogene | 3.36 | 80 |
| | | | family | | |
| 15 | L17131 | HMGIY | high-mobility group (nonhistone | 4.02 | 80 |
| | | | chromosomal) protein isoforms I | | |
| | | | and Y | | |
| 16 | X06323 | MRPL3 | mitochondrial ribosomal protein L3 | 2.93 | 80 |
| 17 | E02628 | | cDNA cording for human | 3.2 | 80 |
| | | | polypeptide chain elongation | | |
| | | | factor-1 alpha | | |
| 18 | D63874 | HMG1 | high-mobility group (nonhistone | 3.23 | 80 |
| | | | chromosomal) protein 1 | | |
| 19 | Y00711 | LDHB | lactate dehydrogenase B | 6.47 | 80 |
| 20 | AI076603 | ESTs | ESTs | 3.12 | 80 |
| 21 | M16650 | ODC1 | ornithine decarboxylase 1 | 3.9 | 75 |
| 22 | AA706503 | EEF1A1 | eukaryotic translation elongation | 3.12 | 75 |
| | | | factor 1 alpha 1 | | |
| 23 | M22382 | HSPD1 | heat shock 60 kD protein 1 | 4.6 | 75 |
| | | | (chaperonin) | | |
| 24 | AI087287 | NOLA2 | nucleolar protein family A, member | 3 | 75 |
| | | | 2 (H/ACA small nucleolar RNPs) | | |
| 25 | S79522 | RPS27A | ribosomal protein S27a | 2.6 | 75 |
| 26 | AI300002 | CCNI | cyclin I | 2.96 | 75 |
| 27 | R12013 | HDCMC04P | hypothetical protein HDCMC04P | 3.33 | 75 |
| 28 | L06132 | VDAC1 | voltage-dependent anion channel 1 | 2.77 | 70 |
| 29 | U66818 | UBE2I | ubiquitin-conjugating enzyme E2I | 3.19 | 70 |
| | | | (homologous to yeast UBC9) | | |
| 30 | J04208 | IMPDH2 | IMP (inisine monophosphate) | 2.81 | 70 |
| | | | dehydrogenase 2 | | |
| 31 | D56784 | DEK | DEK oncogene (DNA binding) | 3.15 | 70 |
| 32 | AA676585 | NPM1 | nucleophosmin (nucleolar | 5.03 | 70 |
| | | | phosphoprotein B23, numatrin) | | |
| 33 | D17554 | RPL6 | ribosomal protein L6 | 3.46 | 70 |
| 34 | U09953 | RPL9 | ribosomal protein L9 | 3.7 | 70 |
| 35 | M77234 | RPS3A | ribosomal protein S3A | 3.25 | 70 |
| 36 | AA916688 | BRF1 | butyrate response factor 1 (EGF- | 2.81 | 70 |
| | | | response factor 1) | | |
| 37 | AA714394 | HMG2 | high-mobility group (nonhistone | 4.54 | 70 |
| | | | chromosomal) protein 2 | | |
| 38 | AA579959 | CYP2S1 | cytochrome P540 family member | 4.03 | 70 |
| | | | predicted from ESTs | | |
| 39 | AI582493 | EST | EST | 3.28 | 70 |
| 40 | X63629 | CDH3 | cadherin 3, type 1, P-cadherin | 3.59 | 65 |
| | | | (placental) | | |
| 41 | AA634090 | HNRPA1 | heterogeneous nuclear | 3.72 | 65 |
| | | | ribonucleoprotein A1 | | |
| 42 | X17206 | RPS2 | ribosomal protein S2 | 3.96 | 65 |
| 43 | K00558 | K-ALPHA | tubulin, alpha, ubiquitous | 3.05 | 60 |
| 44 | D87666 | HSCPA | heat shock 90 kD protein 1, alpha | 2.37 | 60 |
| 45 | N30179 | PLAB | prostate differentiation factor | 7.5 | 60 |
| 46 | L15533 | PAP | pancreatitis-associated protein | 10.04 | 55 |
| 47 | D51696 | TUBB | tubulin, beta polypeptide | 2.96 | 55 |
| 48 | X73460 | RPL3 | ribosomal protein L3 | 2.36 | 55 |
| 49 | M65254 | PPP2R1B | protein phosphatase 2 (formerly | 2.21 | 55 |
| | | | 2A), regulatory subunit A (PR 65), | | |
| | | | beta isoform | | |
| 50 | AW511361 | SLC29A1 | solute carrier family 29 (nucleoside | 4.29 | 55 |
| | | | transporters), member 1 | | |
| 51 | AI291596 | PIGPC1 | p53-induced protein PIGPC1 | 2.59 | 55 |

Among the 5 1 genes, 19 were involved in RNA/protein processing; e.g. ribosomes, translation elongation/initiation factors, and chaperonins. Other up-regulated genes detected included oncogenes (HMGIY, DEK and NPM1), genes encoding cell adhesion/cytoskeleton molecules (TUBB, K-ALPHA, TGFBI, CDH3 and PAP), genes involved in growth control (IMPDH2 and ODC1), signal transduction (BRF1, PLAB, LAP18, CD81 and MACMARCKS), and cell-cycle control (RAN and UBE2I); transcription factors (HMG1 and HMG2), tumor-associated molecules (PPP2R1B, LDHB and SLC29A1), and others.

Down-Regulated Genes

Next, 376 genes (including 127 expressed sequence tags) were identified as consistently down-regulated in both types of tumor by the criteria described above. This group includes genes associated with programmed cell death (CASP8, CASP9, CFLAR, DFFA, PA WR, TNF, TNFRSF10C and TNFRSF12), immunity (chemokine receptors such as IL1RL2, IL17R and IL3RA), growth suppression (Suppressin, DCN, MADH2 and SST), and tumor suppression (TP53). Other down-regulated genes encode cell adhesion/cytoskeleton molecules (e.g. ADAM8, AVL, CDH17, CEACAM1, CTNNA2, ICAPA, KRT9, and ARHGAP5), various metabolic factors (e.g. BPHL, CA2, CA5A, HSD11B2 and ECHS1), ion transporters (SLC15A2, SLC22A1, SLC4A3 and SLC5A1), a nataral antimicrobial molecule (DEFA6), and others. Metabolic enzymes and ion-transport mediators are key factors for maintaining pivotal cellular functions such as detoxication (CA2, CA5A and BPHL) and acid-base balance. Down-regulation of these genes indicates a disruption of cellular homeostasis in tumors (Lawrence et al., 2001).

The list of genes commonly down-regulated in colorectal tumors is set forth in Table 2 below:

TABLE 2
Genes commonly down-regulated in colonic tumors

| | | | | | (Cy3/Cy5 < 0.5), | |
|---|---|---|---|---|---|---|
| | ACCESSION | | | (Cy3/Cy5) | %, | down- |
| No. | No. | GENE | DESCRIPTION | ave | n = 20 | TP |
| 1 | D49817 | PFKFB3 | 6-phosphofructo-2- | 0.2 | 80 | 16 |
| | | | kinase/fructose-2,6- | | | |
| | | | biphosphatase 3 | | | |
| 2 | M55040 | ACHE | acetylcholinesterase (YT | 0.2 | 75 | 15 |
| | | | blood group) | | | |
| 3 | AI052697 | AP1G2 | adaptor-related protein | 0.2 | 90 | 18 |
| | | | complex 1, gamma 2 subunit | | | |
| 4 | J03037 | CA2 | carbonic anhydrase II | 0.2 | 85 | 17 |
| 5 | M98331 | DEFA6 | defensin, alpha 6, Paneth | 0.2 | 75 | 15 |
| | | | cell-specific | | | |
| 6 | J04058 | ETFA | electron-transfer- | 0.2 | 70 | 14 |
| | | | flavoprotein, alpha | | | |
| | | | polypeptide (glutaric aciduria | | | |
| | | | II) | | | |
| 7 | M83941 | EPHA3 | EphA3 | 0.2 | 100 | 20 |
| 8 | M34057 | LTBP1 | latent transforming growth | 0.2 | 75 | 15 |
| | | | factor beta binding protein 1 | | | |
| 9 | AF014923 | LILRA3 | leukocyte immunoglobulin- | 0.2 | 75 | 15 |
| | | | like receptor, subfamily A | | | |
| | | | (without TM domain), | | | |
| | | | member 3 | | | |
| 10 | U10689 | MAGEA5 | melanoma antigen, family A, 5 | 0.2 | 80 | 16 |
| 11 | U97584 | PDE4A | phosphodiesterase 4A, | 0.2 | 75 | 15 |
| | | | cAMP-specific (dunce | | | |
| | | | (Drosophila)-homolog | | | |
| | | | phosphodiesterase E2) | | | |
| 12 | AA262548 | PAWR | PRKC, apoptosis, WT1, | 0.2 | 60 | 12 |
| | | | regulator | | | |
| 13 | AB011004 | UAP1 | UDP-N-acteylglucosamine | 0.2 | 100 | 20 |
| | | | pyrophosphorylase 1 | | | |
| 14 | M17254 | ERG | v-ets avian erythroblastosis | 0.2 | 70 | 14 |
| | | | virus E26 oncogene related | | | |
| 15 | Y11094 | WNT8B | wingless-type MMTV | 0.2 | 80 | 16 |
| | | | integration site family, | | | |
| | | | member 8B | | | |
| 16 | AA705840 | | EST | 0.2 | 85 | 17 |
| 17 | AA586930 | | EST | 0.2 | 80 | 16 |
| 18 | AA229955 | | ESTs | 0.2 | 80 | 16 |
| 19 | AA628600 | | ESTs | 0.2 | 75 | 15 |
| 20 | AA385061 | | ESTs | 0.2 | 70 | 14 |
| 21 | AA521288 | | ESTs, Highly similar to | 0.2 | 75 | 15 |
| | | | carbonic anhydrase VB | | | |
| | | | [H. sapiens] | | | |
| 22 | AI091443 | | Homo sapiens cDNA: | 0.2 | 85 | 17 |
| | | | FLJ21425 fis, clone | | | |
| | | | COL04162 | | | |
| 23 | AA633352 | | Homo sapiens cDNA: | 0.2 | 70 | 14 |
| | | | FLJ23067 fis, clone | | | |
| | | | LNG04993 | | | |
| 24 | AI346913 | CLONE24904 | hypothetical protein | 0.2 | 95 | 19 |
| 25 | D26579 | ADAM8 | a disintegrin and | 0.3 | 80 | 16 |
| | | | metalloproteinase domain 8 | | | |
| 26 | M26393 | ACADS | acyl-Coenzyme A | 0.3 | 85 | 17 |
| | | | dehydrogenase, C-2 to C-3 | | | |
| | | | short chain | | | |
| 27 | AA583019 | ACYP2 | acylphosphatase 2, muscle | 0.3 | 95 | 19 |
| | | | type | | | |
| 28 | U02390 | CAP2 | adenylyl cyclase-associated | 0.3 | 85 | 17 |
| | | | protein 2 | | | |
| 29 | J03853 | ADRA2C | adrenergic, alpha-2C-, | 0.3 | 70 | 14 |
| | | | receptor | | | |
| 30 | M80776 | ADRBK1 | adrenergic, beta, receptor | 0.3 | 75 | 15 |
| | | | kinase 1 | | | |
| 31 | AF041449 | AVIL | advillin | 0.3 | 90 | 18 |
| 32 | M12963 | ADH1 | alcohol dehydrogenase 1 | 0.3 | 80 | 16 |
| | | | (class I), alpha polypeptide | | | |
| 33 | J04795 | AKR1B1 | aldo-keto reductase family 1, | 0.3 | 70 | 14 |
| | | | member B1 (aldose | | | |
| | | | reductase) | | | |
| 34 | AI261581 | AGPS | alkylglycerone phosphate | 0.3 | 65 | 13 |
| | | | synthase | | | |
| 35 | Y07701 | NPEPPS | aminopeptidase puromycin | 0.3 | 65 | 13 |
| | | | sensitive | | | |
| 36 | D00097 | APCS | amyloid P component, | 0.3 | 60 | 12 |
| | | | serum | | | |
| 37 | M11567 | ANG | angiogenin, ribonuclease, | 0.3 | 85 | 17 |
| | | | RNase A family, 5 | | | |
| 38 | X69838 | G9A | ankyrin repeat-containing | 0.3 | 80 | 16 |
| | | | protein | | | |
| 39 | U48408 | AQP6 | aquaporin 6, kidney specific | 0.3 | 95 | 19 |
| 40 | D31833 | AVPR1B | arginine vasopressin | 0.3 | 75 | 15 |
| | | | receptor 1B | | | |
| 41 | X52151 | ARSA | arylsulfatase A | 0.3 | 70 | 14 |
| 42 | L05628 | ABCC1 | ATP-binding cassette, sub- | 0.3 | 85 | 17 |
| | | | family C (CFTR/MRP), | | | |
| | | | member 1 | | | |
| 43 | U66879 | BAD | BCL2-antagonist of cell | 0.3 | 80 | 16 |
| | | | death | | | |
| 44 | X81372 | BPHL | biphenyl hydrolase-like | 0.3 | 75 | 15 |
| | | | (serine hydrolase; breast | | | |
| | | | epithelial mucin-associated | | | |
| | | | antigen) | | | |
| 45 | U39817 | BLM | Bloom syndrome | 0.3 | 100 | 20 |
| 46 | U07969 | CDH17 | cadherin 17, LI cadherin | 0.3 | 80 | 16 |
| | | | (liver-intestine) | | | |
| 47 | L37042 | CSNK1A1 | casein kinase 1, alpha 1 | 0.3 | 80 | 16 |
| 48 | AF015450 | CFLAR | CASP8 and FADD-like | 0.3 | 90 | 18 |
| | | | apoptosis regulator | | | |
| 49 | X98173 | CASP8 | caspase 8, apoptosis-related | 0.3 | 95 | 19 |
| | | | cysteine protease | | | |
| 50 | U60521 | CASP9 | caspase 9, apoptosis-related | 0.3 | 95 | 19 |
| | | | cysteine protease | | | |
| 51 | M94151 | CTNNA2 | catenin (cadherin-associated | 0.3 | 80 | 16 |
| | | | protein), alpha 2 | | | |
| 52 | AF013611 | CTSW | cathepsin W (lymphopain) | 0.3 | 70 | 14 |
| 53 | H90902 | CDC23 | CDC23 (cell division cycle | 0.3 | 80 | 16 |
| | | | 23, yeast, homolog) | | | |
| 54 | U67615 | CHS1 | Chediak-Higashi syndrome 1 | 0.3 | 95 | 19 |
| 55 | U54994 | CCR5 | chemokine (C-C motif) | 0.3 | 75 | 15 |
| | | | receptor 5 | | | |
| 56 | M30185 | CETP | cholesteryl ester transfer | 0.3 | 95 | 19 |
| | | | protein, plasma | | | |
| 57 | U20980 | CHAF1B | chromatin assembly factor 1, | 0.3 | 85 | 17 |
| | | | subunit B (p60) | | | |
| 58 | X92098 | RNP24 | coated vesicle membrane | 0.3 | 90 | 18 |
| | | | protein | | | |
| 59 | X70476 | COPB2 | coatomer protein complex, | 0.3 | 95 | 19 |
| | | | subunit beta 2 (beta prime) | | | |
| 60 | L29349 | CSF2RA | colony stimulating factor 2 | 0.3 | 90 | 18 |
| | | | receptor, alpha, low-affinity | | | |
| | | | (granulocyte-macrophage) | | | |
| 61 | M59941 | CSF2RB | colony stimulating factor 2 | 0.3 | 90 | 18 |
| | | | receptor, beta, low-affinity | | | |
| | | | (granulocyte-macrophage) | | | |
| 62 | AI312573 | CPNE3 | copine III | 0.3 | 70 | 14 |
| 63 | R58976 | CORO1C | coronin, actin-binding | 0.3 | 85 | 17 |
| | | | protein, 1C | | | |
| 64 | J03870 | CST1 | cystatin SN | 0.3 | 70 | 14 |
| 65 | X82224 | CCBL1 | cysteine conjugate-beta | 0.3 | 95 | 19 |
| | | | lyase; cytoplasmic | | | |
| | | | (glutamine transaminase K, | | | |
| | | | kyneurenine | | | |
| | | | aminotransferase) | | | |
| 66 | D49738 | CKAP1 | cytoskeleton-associated | 0.3 | 95 | 19 |
| | | | protein 1 | | | |
| 67 | M14219 | DCN | decorin | 0.3 | 85 | 17 |
| 68 | S79854 | DIO3 | deiodinase, iodothyronine, | 0.3 | 100 | 20 |
| | | | type III | | | |
| 69 | L40817 | DNASE1L1 | deoxyribonuclease I-like 1 | 0.3 | 90 | 18 |
| 70 | AI149258 | DKK3 | dickkopf (Xenopus laevis) | 0.3 | 85 | 17 |
| | | | homolog 3 | | | |
| 71 | U91985 | DFFA | DNA fragmentation factor, | 0.3 | 60 | 12 |
| | | | 45 kD, alpha polypeptide | | | |
| 72 | AA252866 | KIP2 | DNA-dependent protein | 0.3 | 85 | 17 |
| | | | kinase catalytic subunit- | | | |
| | | | interacting protein 2 | | | |
| 73 | N63007 | DPM1 | dolichyl-phosphate | 0.3 | 70 | 14 |
| | | | mannosyltransferase | | | |
| | | | polypeptide 1, catalytic | | | |
| | | | subunit | | | |
| 74 | AJ000522 | DNAH17 | dynein, axonemal, heavy | 0.3 | 90 | 18 |
| | | | polypeptide 17 | | | |
| 75 | U03877 | EFEMP1 | EGF-containing fibulin-like | 0.3 | 75 | 15 |
| | | | extracellular matrix protein 1 | | | |
| 76 | AF011466 | EDG4 | endothelial differentiation, | 0.3 | 85 | 17 |
| | | | lysophosphatidic acid G- | | | |
| | | | protein-coupled receptor, 4 | | | |
| 77 | X94553 | FOXE2 | forkhead box E2 | 0.3 | 85 | 17 |
| 78 | U58975 | FRAT1 | frequently rearranged in | 0.3 | 85 | 17 |
| | | | advanced T-cell lymphomas | | | |
| 79 | U63917 | GPR30 | G protein-coupled receptor | 0.3 | 80 | 16 |
| | | | 30 | | | |
| 80 | M84443 | GALK2 | galactokinase 2 | 0.3 | 90 | 18 |
| 81 | M19722 | FGR | Gardner-Rasheed feline | 0.3 | 80 | 16 |
| | | | sarcoma viral (v-fgr) | | | |
| | | | oncogene homolog | | | |
| 82 | X17254 | GATA1 | GATA-binding protein 1 | 0.3 | 85 | 17 |
| | | | (globin transcription factor 1) | | | |
| 83 | D14886 | GTF2A1 | general transcription factor | 0.3 | 85 | 17 |
| | | | IIA, 1 (37 kD and 19 kD | | | |
| | | | subunits) | | | |
| 84 | L19659 | GCNT2 | glucosaminyl (N-acetyl) | 0.3 | 75 | 15 |
| | | | transferase 2, I-branching | | | |
| | | | enzyme | | | |
| 85 | U92459 | GRM8 | glutamate receptor, | 0.3 | 85 | 17 |
| | | | metabotropic 8 | | | |
| 86 | U79725 | GPA33 | glycoprotein A33 | 0.3 | 75 | 15 |
| | | | (transmembrane) | | | |
| 87 | X54101 | GNLY | granulysin | 0.3 | 80 | 16 |
| 88 | M18930 | HPN | hepsin (transmembrane | 0.3 | 95 | 19 |
| | | | protease, serine 1) | | | |
| 89 | AF040714 | HOXA10 | homeo box A10 | 0.3 | 95 | 19 |
| 90 | X61755 | HOXC5 | homeo box C5 | 0.3 | 80 | 16 |
| 91 | U14631 | HSD11B2 | hydroxysteroid (11-beta) | 0.3 | 85 | 17 |
| | | | dehydrogenase 2 | | | |
| 92 | X57206 | ITPKB | inositol 1,4,5-trisphosphate | 0.3 | 75 | 15 |
| | | | 3-kinase B | | | |
| 93 | Y11360 | IMPA1 | inositol(myo)(or 4)- | 0.3 | 95 | 19 |
| | | | monophosphatase 1 | | | |
| 94 | U30329 | IPF1 | insulin promoter factor 1, | 0.3 | 90 | 18 |
| | | | homeodomain transcription | | | |
| | | | factor | | | |
| 95 | AF055028 | IGFBP7 | insulin-like growth factor | 0.3 | 90 | 18 |
| | | | binding protein 7 | | | |
| 96 | AA226073 | ITM2C | integral membrane protein | 0.3 | 95 | 19 |
| | | | 2C | | | |
| 97 | AF012023 | ICAPA | integrin cytoplasmic domain- | 0.3 | 95 | 19 |
| | | | associated protein 1 | | | |
| 98 | M34480 | ITGA2B | integrin, alpha 2b (platelet | 0.3 | 70 | 14 |
| | | | glycoprotein IIb of IIb/IIIa | | | |
| | | | complex, antigen CD41B) | | | |
| 99 | L25851 | ITGAE | integrin, alpha E (antigen | 0.3 | 95 | 19 |
| | | | CD103, human mucosal | | | |
| | | | lymphocyte antigen 1; alpha | | | |
| | | | polypeptide) | | | |
| 100 | Y00796 | ITGAL | integrin, alpha L (antigen | 0.3 | 90 | 18 |
| | | | CD11A (p180), lymphocyte | | | |
| | | | function-associated antigen | | | |
| | | | 1; alpha polypeptide) | | | |
| 101 | Z56281 | IRF3 | interferon regulatory factor 3 | 0.3 | 90 | 18 |
| 102 | U49065 | IL1RL2 | interleukin 1 receptor-like 2 | 0.3 | 90 | 18 |
| 103 | U03187 | IL12RB1 | interleukin 12 receptor, beta 1 | 0.3 | 75 | 15 |
| 104 | M74782 | IL3RA | interleukin 3 receptor, alpha | 0.3 | 75 | 15 |
| | | | (low affinity) | | | |
| 105 | L01100 | ICA1 | islet cell autoantigen 1 | 0.3 | 70 | 14 |
| | | | (69 kD) | | | |
| 106 | X55885 | KDELR1 | KDEL (Lys-Asp-Glu-Leu) | 0.3 | 80 | 16 |
| | | | endoplasmic reticulum | | | |
| | | | protein retention receptor 1 | | | |
| 107 | Z29074 | KRT9 | keratin 9 (epidermolytic | 0.3 | 70 | 14 |
| | | | palmoplantar keratoderma) | | | |
| 108 | AF028840 | LOC51045 | Kruppel-associated box | 0.3 | 80 | 16 |
| | | | protein | | | |
| 109 | U69566 | LATS2 | LATS (large tumor | 0.3 | 85 | 17 |
| | | | suppressor, Drosophila) | | | |
| | | | homolog 2 | | | |
| 110 | AA779658 | LEPR | leptin receptor | 0.3 | 80 | 16 |
| 111 | D50532 | HML2 | macrophage lectin 2 | 0.3 | 90 | 18 |
| | | | (calcium dependent) | | | |
| 112 | AA628220 | MBL1P1 | mannose-binding lectin | 0.3 | 90 | 18 |
| | | | (protein A) 1, pseudogene 1 | | | |
| 113 | AF056334 | MAGEC1 | melanoma antigen, family C, 1 | 0.3 | 80 | 16 |
| 114 | M65131 | MUT | methylmalonyl Coenzyme A | 0.3 | 75 | 15 |
| | | | mutase | | | |
| 115 | X96698 | METTL1 | methyltransferase-like 1 | 0.3 | 95 | 19 |
| 116 | U77129 | MAP4K5 | mitogen-activated protein | 0.3 | 100 | 20 |
| | | | kinase kinase kinase kinase 5 | | | |
| 117 | AF060154 | MSC | musculin (activated B-cell | 0.3 | 90 | 18 |
| | | | factor) | | | |
| 118 | U75330 | NCAM2 | neural cell adhesion | 0.3 | 95 | 19 |
| | | | molecule 2 | | | |
| 119 | U77968 | NPAS1 | neuronal PAS domain | 0.3 | 60 | 12 |
| | | | protein 1 | | | |
| 120 | U17989 | GS2NA | nuclear autoantigen | 0.3 | 80 | 16 |
| 121 | U16258 | NFKBIL2 | nuclear factor of kappa light | 0.3 | 90 | 18 |
| | | | polypeptide gene enhancer | | | |
| | | | in B-cells inhibitor-like 2 | | | |
| 122 | U15306 | NFX1 | nuclear transcription factor, | 0.3 | 80 | 16 |
| | | | X-box binding 1 | | | |
| 123 | U30185 | OPRL1 | opiate receptor-like 1 | 0.3 | 75 | 15 |
| 124 | U42387 | PPYR1 | pancreatic polypeptide | 0.3 | 95 | 19 |
| | | | receptor 1 | | | |
| 125 | L40401 | ZAP128 | peroxisomal long-chain acyl- | 0.3 | 90 | 18 |
| | | | coA thioesterase; putative | | | |
| | | | protein | | | |
| 126 | AI188655 | PDCL | phosducin-like | 0.3 | 75 | 15 |
| 127 | U03090 | PLA2G5 | phospholipase A2, group V | 0.3 | 95 | 19 |
| 128 | U07364 | KCNJ4 | potassium inwardly- | 0.3 | 85 | 17 |
| | | | rectifying channel, subfamily | | | |
| | | | J, member 4 | | | |
| 129 | AI341177 | PPARBP | PPAR binding protein | 0.3 | 85 | 17 |
| 130 | AJ001810 | CFIM25 | pre-mRNA cleavage factor | 0.3 | 85 | 17 |
| | | | lm (25 kD) | | | |
| 131 | AF009243 | PRRG2 | proline-rich Gla (G- | 0.3 | 90 | 18 |
| | | | carboxyglutamic acid) | | | |
| | | | polypeptide 2 | | | |
| 132 | AI357236 | PRM1 | protamine 1 | 0.3 | 80 | 16 |
| 133 | AB003177 | PSMD9 | proteasome (prosome, | 0.3 | 80 | 16 |
| | | | macropain) 26S subunit, | | | |
| | | | non-ATPase, 9 | | | |
| 134 | AA532845 | P5 | protein disulfide isomerase- | 0.3 | 80 | 16 |
| | | | related protein | | | |
| 135 | U48250 | PRKCBP2 | protein kinase C binding | 0.3 | 80 | 16 |
| | | | protein 2 | | | |
| 136 | X80910 | PPP1CB | protein phosphatase 1, | 0.3 | 90 | 18 |
| | | | catalytic subunit, beta | | | |
| | | | isoform | | | |
| 137 | U26446 | PPOX | protoporphyrinogen oxidase | 0.3 | 95 | 19 |
| 138 | X83688 | P2RX1 | purinergic receptor P2X, | 0.3 | 90 | 18 |
| | | | ligand-gated ion channel, 1 | | | |
| 139 | D38449 | GPR | putative G protein coupled | 0.3 | 75 | 15 |
| | | | receptor | | | |
| 140 | AA196253 | RAD51C | RAD51 (S. cerevisiae) | 0.3 | 75 | 15 |
| | | | homolog C | | | |
| 141 | L08010 | REG1B | regenerating islet-derived 1 | 0.3 | 90 | 18 |
| | | | beta (pancreatic stone | | | |
| | | | protein, pancreatic thread | | | |
| | | | protein) | | | |
| 142 | AF073710 | RGS9 | regulator of G-protein | 0.3 | 75 | 15 |
| | | | signalling 9 | | | |
| 143 | AF012270 | RRH | retinal pigment epithelium- | 0.3 | 80 | 16 |
| | | | derived rhodopsin homolog | | | |
| 144 | U17032 | ARHGAP5 | Rho GTPase activating | 0.3 | 75 | 15 |
| | | | protein 5 | | | |
| 145 | AL009266 | RBM9 | RNA binding motif protein 9 | 0.3 | 70 | 14 |
| 146 | D28483 | RBMS2 | RNA binding motif, single | 0.3 | 85 | 17 |
| | | | stranded interacting protein 2 | | | |
| 147 | AI161013 | S100A12 | S100 calcium-binding | 0.3 | 85 | 17 |
| | | | protein A12 (calgranulin C) | | | |
| 148 | AF031920 | SGCE | sarcoglycan, epsilon | 0.3 | 70 | 14 |
| 149 | M90439 | SERPINF1 | serine (or cysteine) | 0.3 | 85 | 17 |
| | | | proteinase inhibitor, clade F | | | |
| | | | (alpha-2 antiplasmin, | | | |
| | | | pigment epithelium derived | | | |
| | | | factor), member 1 | | | |
| 150 | U59305 | PK428 | Ser-Thr protein kinase | 0.3 | 85 | 17 |
| | | | related to the myotonic | | | |
| | | | dystrophy protein kinase | | | |
| 151 | U71383 | SIGLEC5 | sialic acid binding lg-like | 0.3 | 90 | 18 |
| | | | lectin 5 | | | |
| 152 | L41142 | STAT5A | signal transducer and | 0.3 | 70 | 14 |
| | | | activator of transcription 5A | | | |
| 153 | AA521019 | SNRPG | small nuclear | 0.3 | 85 | 17 |
| | | | ribonucleoprotein | | | |
| | | | polypeptide G | | | |
| 154 | AA084871 | YKT6 | SNARE protein | 0.3 | 85 | 17 |
| 155 | M81758 | SCN4A | sodium channel, voltage- | 0.3 | 75 | 15 |
| | | | gated, type IV, alpha | | | |
| | | | polypeptide | | | |
| 156 | S78203 | SLC15A2 | solute carrier family 15 | 0.3 | 85 | 17 |
| | | | (H+/peptide transporter), | | | |
| | | | member 2 | | | |
| 157 | U77086 | SLC22A1 | solute carrier family 22 | 0.3 | 80 | 16 |
| | | | (organic cation transporter), | | | |
| | | | member 1 | | | |
| 158 | AA401224 | SLC25A14 | solute carrier family 25 | 0.3 | 80 | 16 |
| | | | (mitochondrial carrier, brain), | | | |
| | | | member 14 | | | |
| 159 | L02785 | SLC26A3 | solute carrier family 26, | 0.3 | 80 | 16 |
| | | | member 3 | | | |
| 160 | U05596 | SLC4A3 | solute carrier family 4, anion | 0.3 | 75 | 15 |
| | | | exchanger, member 3 | | | |
| 161 | Y16610 | SPG7 | spastic paraplegia 7, | 0.3 | 95 | 19 |
| | | | paraplegin (pure and | | | |
| | | | complicated autosomal | | | |
| | | | recessive) | | | |
| 162 | AA889336 | SPAG6 | sperm associated antigen 6 | 0.3 | 85 | 17 |
| 163 | AJ222801 | SMPD2 | sphingomyelin | 0.3 | 80 | 16 |
| | | | phosphodiesterase 2, | | | |
| | | | neutral membrane (neutral | | | |
| | | | sphingomyelinase) | | | |
| 164 | AF055460 | STC2 | stanniocalcin 2 | 0.3 | 95 | 19 |
| 165 | X63597 | SI | sucrase-isomaltase | 0.3 | 80 | 16 |
| 166 | AF007165 | SPN | suppressin (nuclear | 0.3 | 75 | 15 |
| | | | deformed epidermal | | | |
| | | | autoregulatory factor | | | |
| | | | (DEAF)-related) | | | |
| 167 | L10123 | SPAR | surfactant protein A binding | 0.3 | 85 | 17 |
| | | | protein | | | |
| 168 | AF009039 | SYNJ1 | synaptojanin 1 | 0.3 | 85 | 17 |
| 169 | AI348910 | STX10 | syntaxin 10 | 0.3 | 85 | 17 |
| 170 | AF004562 | STXBP1 | syntaxin binding protein 1 | 0.3 | 100 | 20 |
| 171 | U03399 | TCP10 | t-complex 10 (a murine tcp | 0.3 | 70 | 14 |
| | | | homolog) | | | |
| 172 | D29767 | TEC | tec protein tyrosine kinase | 0.3 | 85 | 17 |
| 173 | U86136 | TEP1 | telomerase-associated | 0.3 | 95 | 19 |
| | | | protein 1 | | | |
| 174 | X59434 | TST | thiosulfate sulfurtransferase | 0.3 | 90 | 18 |
| | | | (rhodanese) | | | |
| 175 | D38081 | TBXA2R | thromboxane A2 receptor | 0.3 | 65 | 13 |
| 176 | AI142918 | TJP3 | tight junction protein 3 (zona | 0.3 | 80 | 16 |
| | | | occludens 3) | | | |
| 177 | AF007872 | TOR1B | torsin family 1, member B | 0.3 | 95 | 19 |
| | | | (torsin B) | | | |
| 178 | U85658 | TFAP2C | transcription factor AP-2 | 0.3 | 90 | 18 |
| | | | gamma (activating | | | |
| | | | enhancer-binding protein 2 | | | |
| | | | gamma) | | | |
| 179 | AA215687 | TACC3 | transforming, acidic coiled- | 0.3 | 100 | 20 |
| | | | coil containing protein 3 | | | |
| 180 | D10653 | TM4SF2 | transmembrane 4 | 0.3 | 85 | 17 |
| | | | superfamily member 2 | | | |
| 181 | M16441 | TNF | tumor necrosis factor (TNF | 0.3 | 85 | 17 |
| | | | superfamily, member 2) | | | |
| 182 | AF012536 | TNFRSF10C | tumor necrosis factor | 0.3 | 95 | 19 |
| | | | receptor superfamily, | | | |
| | | | member 10c, decoy without | | | |
| | | | an intracellular domain | | | |
| 183 | U43408 | TNK1 | tyrosine kinase, non- | 0.3 | 65 | 13 |
| | | | receptor, 1 | | | |
| 184 | D49676 | U2AF1RS1 | U2 small nuclear | 0.3 | 90 | 18 |
| | | | ribonucleoprotein auxiliary | | | |
| | | | factor, small subunit 1 | | | |
| 185 | AF079564 | USP2 | ubiquitin specific protease 2 | 0.3 | 65 | 13 |
| 186 | AA703115 | USP24 | ubiquitin specific protease | 0.3 | 70 | 14 |
| | | | 24 | | | |
| 187 | AA702803 | BM039 | uncharacterized bone | 0.3 | 80 | 16 |
| | | | marrow protein BM039 | | | |
| 188 | AI081684 | VNN1 | vanin 1 | 0.3 | 100 | 20 |
| 189 | AA912674 | VE-JAM | vascular endothelial | 0.3 | 90 | 18 |
| | | | junction-associated | | | |
| | | | molecule | | | |
| 190 | L13288 | VIPR1 | vasoactive intestinal peptide | 0.3 | 75 | 15 |
| | | | receptor 1 | | | |
| 191 | X15218 | SKI | v-ski avian sarcoma viral | 0.3 | 75 | 15 |
| | | | oncogene homolog | | | |
| 192 | U12707 | WAS | Wiskott-Aldrich syndrome | 0.3 | 95 | 19 |
| | | | (eczema-thrombocytopenia) | | | |
| 193 | U66561 | ZNF184 | zinc finger protein 184 | 0.3 | 80 | 16 |
| | | | (Kruppel-like) | | | |
| 194 | U95044 | ZNF230 | zinc finger protein 230 | 0.3 | 80 | 16 |
| 195 | U40462 | ZNFN1A1 | zinc finger protein, subfamily | 0.3 | 80 | 16 |
| | | | 1A, 1 (Ikaros) | | | |
| 196 | AA614419 | | EST | 0.3 | 90 | 18 |
| 197 | AA744242 | | EST | 0.3 | 90 | 18 |
| 198 | AA757990 | | EST | 0.3 | 85 | 17 |
| 199 | AA758080 | | EST | 0.3 | 85 | 17 |
| 200 | AA634552 | | EST | 0.3 | 85 | 17 |
| 201 | AA700554 | | EST | 0.3 | 80 | 16 |
| 202 | AA609289 | | EST | 0.3 | 80 | 16 |
| 203 | AA747958 | | EST | 0.3 | 75 | 15 |
| 204 | AA626369 | | EST | 0.3 | 85 | 17 |
| 205 | AA758302 | | EST | 0.3 | 85 | 17 |
| 206 | AA609343 | | EST | 0.3 | 80 | 16 |
| 207 | AA663323 | | EST | 0.3 | 85 | 17 |
| 208 | AA709224 | | EST | 0.3 | 70 | 14 |
| 209 | AA625955 | | EST | 0.3 | 65 | 13 |
| 210 | AA442345 | | ESTs | 0.3 | 100 | 20 |
| 211 | R10153 | | ESTs | 0.3 | 95 | 19 |
| 212 | AA759254 | | ESTs | 0.3 | 95 | 19 |
| 213 | AA829640 | | ESTs | 0.3 | 95 | 19 |
| 214 | AA034063 | | ESTs | 0.3 | 90 | 18 |
| 215 | W88544 | | ESTs | 0.3 | 90 | 18 |
| 216 | AA827701 | | ESTs | 0.3 | 90 | 18 |
| 217 | T79190 | | ESTs | 0.3 | 85 | 17 |
| 218 | H16240 | | ESTs | 0.3 | 95 | 19 |
| 219 | R44603 | | ESTs | 0.3 | 85 | 17 |
| 220 | AA694517 | | ESTs | 0.3 | 85 | 17 |
| 221 | AA620749 | | ESTs | 0.3 | 85 | 17 |
| 222 | W95087 | | ESTs | 0.3 | 85 | 17 |
| 223 | AA495917 | | ESTs | 0.3 | 85 | 17 |
| 224 | W39642 | | ESTs | 0.3 | 85 | 17 |
| 225 | N67031 | | ESTs | 0.3 | 85 | 17 |
| 226 | AA758209 | | ESTs | 0.3 | 80 | 16 |
| 227 | AA702399 | | ESTs | 0.3 | 80 | 16 |
| 228 | R41450 | | ESTs | 0.3 | 90 | 18 |
| 229 | AA634446 | | ESTs | 0.3 | 90 | 18 |
| 230 | R84284 | | ESTs | 0.3 | 85 | 17 |
| 231 | AA534321 | | ESTs | 0.3 | 85 | 17 |
| 232 | N63761 | | ESTs | 0.3 | 85 | 17 |
| 233 | AA427610 | | ESTs | 0.3 | 85 | 17 |
| 234 | R07651 | | ESTs | 0.3 | 80 | 16 |
| 235 | AA002191 | | ESTs | 0.3 | 75 | 15 |
| 236 | AA701558 | | ESTs | 0.3 | 75 | 15 |
| 237 | H70098 | | ESTs | 0.3 | 80 | 16 |
| 238 | H04150 | | ESTs | 0.3 | 80 | 16 |
| 239 | U55988 | | ESTs | 0.3 | 75 | 15 |
| 240 | R00186 | | ESTs | 0.3 | 70 | 14 |
| 241 | AI057128 | | ESTs | 0.3 | 70 | 14 |
| 242 | AA481948 | | ESTs | 0.3 | 70 | 14 |
| 243 | R19897 | | ESTs | 0.3 | 65 | 13 |
| 244 | AA804409 | | ESTs | 0.3 | 75 | 15 |
| 245 | AA602585 | | ESTs | 0.3 | 75 | 15 |
| 246 | AI078363 | | ESTs | 0.3 | 70 | 14 |
| 247 | AI299327 | | ESTs | 0.3 | 65 | 13 |
| 248 | H15165 | | ESTs | 0.3 | 70 | 14 |
| 249 | AA453726 | | ESTs | 0.3 | 65 | 13 |
| 250 | AA427737 | | ESTs | 0.3 | 65 | 13 |
| 251 | AA669139 | | ESTs | 0.3 | 70 | 14 |
| 252 | AA743399 | | ESTs | 0.3 | 70 | 14 |
| 253 | AA019167 | | ESTs | 0.3 | 65 | 13 |
| 254 | AI344053 | | ESTs | 0.3 | 65 | 13 |
| 255 | AI224893 | | ESTs, Highly similar to | 0.3 | 75 | 15 |
| | | | KJHUAB N- | | | |
| | | | acetylgalactosamine-4- | | | |
| | | | sulfatase [H. sapiens] | | | |
| 256 | AA634543 | | ESTs, Moderately similar to | 0.3 | 80 | 16 |
| | | | IGF-II mRNA-binding protein | | | |
| | | | 3 [H. sapiens] | | | |
| 257 | AI222465 | | ESTs, Moderately similar to | 0.3 | 80 | 16 |
| | | | JC1235 transcription factor | | | |
| | | | BTF3a [H. sapiens] | | | |
| 258 | AA478781 | | ESTs, Weakly similar to | 0.3 | 65 | 13 |
| | | | similar to yeast adenylate | | | |
| | | | cyclase [H. sapiens] | | | |
| 259 | AA612666 | | Homo sapiens BAC clone | 0.3 | 95 | 19 |
| | | | 215O12 NG35, NG36, G9A, | | | |
| | | | NG22, G9, HSP70-2, | | | |
| | | | HSP70, HSP70-HOM, | | | |
| | | | snRNP, G7A, NG37, NG23, | | | |
| | | | and MutSH5 genes, | | | |
| | | | complete cds | | | |
| 260 | AA565741 | | Homo sapiens cDNA | 0.3 | 95 | 19 |
| | | | FLJ10131 fis, clone | | | |
| | | | HEMBA1003041 | | | |
| 261 | N74014 | | Homo sapiens cDNA | 0.3 | 80 | 16 |
| | | | FLJ12229 fis, clone | | | |
| | | | MAMMA1001181, weakly | | | |
| | | | similar to ABC1 PROTEIN | | | |
| | | | HOMOLOG PRECURSOR | | | |
| 262 | D81580 | | Homo sapiens cDNA | 0.3 | 85 | 17 |
| | | | FLJ12782 fis, clone | | | |
| | | | NT2RP2001869, moderately | | | |
| | | | similar to ZINC FINGER | | | |
| | | | PROTEIN 191 | | | |
| 263 | AA292001 | | Homo sapiens cDNA: | 0.3 | 90 | 18 |
| | | | FLJ22251 fis, clone | | | |
| | | | HRC02686 | | | |
| 264 | AI123509 | | Homo sapiens cDNA: | 0.3 | 85 | 17 |
| | | | FLJ23107 fis, clone | | | |
| | | | LNG07738 | | | |
| 265 | AF063725 | | Homo sapiens clone | 0.3 | 70 | 14 |
| | | | BCSynL38 immunoglobulin | | | |
| | | | lambda light chain variable | | | |
| | | | region mRNA, partial cds | | | |
| 266 | AA430290 | | Homo sapiens mRNA; cDNA | 0.3 | 90 | 18 |
| | | | DKFZp434C1717 (from | | | |
| | | | clone DKFZp434C1717); | | | |
| | | | partial cds | | | |
| 267 | H98096 | | Homo sapiens mRNA; cDNA | 0.3 | 85 | 17 |
| | | | DKFZp434M196 (from clone | | | |
| | | | DKFZp434M196) | | | |
| 268 | N48361 | | Homo sapiens mRNA; cDNA | 0.3 | 85 | 17 |
| | | | DKFZp564O1016 (from | | | |
| | | | clone DKFZp564O1016) | | | |
| 269 | AI365683 | | Homo sapiens PAC clone | 0.3 | 80 | 16 |
| | | | RP4-751H13 from 7q35-qter | | | |
| 270 | N80334 | DKFZP586O0223 | hypothetical protein | 0.3 | 85 | 17 |
| 271 | AA788772 | DKFZp762B226 | hypothetical protein | 0.3 | 70 | 14 |
| | | | DKFZp762B226 | | | |
| 272 | R05487 | FLJ10955 | hypothetical protein | 0.3 | 75 | 15 |
| | | | FLJ10955 | | | |
| 273 | AA825485 | FLJ13163 | hypothetical protein | 0.3 | 85 | 17 |
| | | | FLJ13163 | | | |
| 274 | AA620802 | FLJ20284 | hypothetical protein | 0.3 | 100 | 20 |
| | | | FLJ20284 | | | |
| 275 | AA037467 | KIAA1165 | hypothetical protein | 0.3 | 70 | 14 |
| | | | KIAA1165 | | | |
| 276 | D13645 | KIAA0020 | KIAA0020 gene product | 0.3 | 75 | 15 |
| 277 | AF055995 | KIAA0130 | KIAA0130 gene product | 0.3 | 75 | 15 |
| 278 | AA354387 | KIAA0285 | KIAA0285 gene product | 0.3 | 85 | 17 |
| 279 | AI167403 | KIAA0828 | KIAA0828 protein | 0.3 | 85 | 17 |
| 280 | AI285498 | KIAA0962 | KIAA0962 protein | 0.3 | 80 | 16 |
| 281 | AA477862 | KIAA0974 | KIAA0974 protein | 0.3 | 85 | 17 |
| 282 | W84743 | KIAA1203 | KIAA1203 protein | 0.3 | 70 | 14 |
| 283 | AA234909 | KIAA1306 | KIAA1306 protein | 0.3 | 80 | 16 |
| 284 | AA503387 | KIAA1357 | KIAA1357 protein | 0.3 | 85 | 17 |
| 285 | U90919 | LOC57862 | clones 23667 and 23775 | 0.3 | 100 | 20 |
| | | | zinc finger protein | | | |
| 286 | AA426093 | LOC57862 | clones 23667 and 23775 | 0.3 | 95 | 19 |
| | | | zinc finger protein | | | |
| 287 | AA311912 | DKFZP564A122 | DKFZP564A122 protein | 0.3 | 75 | 15 |
| 288 | AI089622 | LOC51025 | CGI36 protein | 0.3 | 85 | 17 |
| 289 | AA040452 | LOC51118 | CGI-94 protein | 0.3 | 70 | 14 |
| 290 | X68487 | ADORA2B | adenosine A2b receptor | 0.4 | 80 | 16 |
| 291 | H04112 | ALG5 | Alg5, S. cerevisiae, homolog | 0.4 | 85 | 17 |
| | | | of | | | |
| 292 | AF070598 | ABCB6 | ATP-binding cassette, sub- | 0.4 | 80 | 16 |
| | | | family B (MDR/TAP), | | | |
| | | | member 6 | | | |
| 293 | AA150869 | ADIR | ATP-dependant interferon | 0.4 | 65 | 13 |
| | | | response protein 1 | | | |
| 294 | U61538 | CHP | calcium binding protein P22 | 0.4 | 85 | 17 |
| 295 | U07139 | CACNB3 | calcium channel, voltage- | 0.4 | 85 | 17 |
| | | | dependent beta 3 subunit | | | |
| 296 | L19297 | CA5A | carbonic anhydrase.VA, | 0.4 | 60 | 12 |
| | | | mitochondrial | | | |
| 297 | X16354 | CEACAM1 | carcinoembryonic antigen- | 0.4 | 90 | 18 |
| | | | related cell adhesion | | | |
| | | | molecule 1 (biliary | | | |
| | | | glycoprotein) | | | |
| 298 | L39211 | CPT1A | carnitine | 0.4 | 65 | 13 |
| | | | palmitoyltransferase I, liver | | | |
| 299 | X59350 | CD22 | CD22 antigen | 0.4 | 75 | 15 |
| 300 | D13305 | CCKBR | cholecystokinin B receptor | 0.4 | 65 | 13 |
| 301 | D13900 | ECHS1 | enoyl Coenzyme A | 0.4 | 75 | 15 |
| | | | hydratase, short chain, 1, | | | |
| | | | mitochondrial | | | |
| 302 | U11690 | FGD1 | faciogenital dysplasia | 0.4 | 85 | 17 |
| | | | (Aarskog-Scott syndrome) | | | |
| 303 | U10991 | G2 | G2 protein | 0.4 | 75 | 15 |
| 304 | U03486 | GJA5 | gap junction protein, alpha | 0.4 | 75 | 15 |
| | | | 5, 40 kD (connexin 40) | | | |
| 305 | U01156 | GLP1R | glucagon-like peptide 1 | 0.4 | 80 | 16 |
| | | | receptor | | | |
| 306 | X92518 | HMGIC | high-mobility group | 0.4 | 75 | 15 |
| | | | (nonhistone chromosomal) | | | |
| | | | protein isoform I-C | | | |
| 307 | AF039691 | HDAC5 | histone deacetylase 5 | 0.4 | 75 | 15 |
| 308 | U79734 | HIP1 | huntingtin interacting protein 1 | 0.4 | 65 | 13 |
| 309 | M65291 | IL12A | interleukin 12A (natural killer | 0.4 | 75 | 15 |
| | | | cell stimulatory factor 1, | | | |
| | | | cytotoxic lymphocyte | | | |
| | | | maturation factor 1, p35) | | | |
| 310 | U58917 | IL17R | interleukin 17 receptor | 0.4 | 65 | 13 |
| 311 | AF005361 | KPNA5 | karyopherin alpha 5 | 0.4 | 65 | 13 |
| | | | (importin alpha 6) | | | |
| 312 | U59911 | MADH2 | MAD (mothers against | 0.4 | 70 | 14 |
| | | | decapentaplegic, | | | |
| | | | Drosophila) homolog 2 | | | |
| 313 | U37248 | MAN2C1 | mannosidase, alpha, class | 0.4 | 80 | 16 |
| | | | 2C, member 1 | | | |
| 314 | AF053551 | MTX2 | metaxin 2 | 0.4 | 90 | 18 |
| 315 | X80199 | MLN51 | MLN51 protein | 0.4 | 80 | 16 |
| 316 | U88573 | NBR2 | NBR2 | 0.4 | 90 | 18 |
| 317 | U22662 | NR1H3 | nuclear receptor subfamily | 0.4 | 85 | 17 |
| | | | 1, group H, member 3 | | | |
| 318 | L25597 | PAX2 | paired box gene 2 | 0.4 | 85 | 17 |
| 319 | AF069301 | PECI | peroxisomal D3,D2-enoyl- | 0.4 | 75 | 15 |
| | | | CoA isomerase | | | |
| 320 | AF014402 | PPAP2A | phosphatidic acid | 0.4 | 90 | 18 |
| | | | phosphatase type 2A | | | |
| 321 | K03021 | PLAT | plasminogen activator, | 0.4 | 70 | 14 |
| | | | tissue | | | |
| 322 | T84015 | PLEC1 | plectin 1, intermediate | 0.4 | 65 | 13 |
| | | | filament binding protein, | | | |
| | | | 500 kD | | | |
| 323 | AI188134 | POLA2 | polymerase (DNA-directed), | 0.4 | 65 | 13 |
| | | | alpha (70 kD) | | | |
| 324 | U10099 | POMZP3 | POM (POM121 rat homolog) | 0.4 | 85 | 17 |
| | | | and ZP3 fusion protein | | | |
| 325 | Z11898 | POU5F1 | POU domain, class 5, | 0.4 | 70 | 14 |
| | | | transcription factor 1 | | | |
| 326 | R26785 | PRSS8 | protease, serine, 8 | 0.4 | 60 | 12 |
| | | | (prostasin) | | | |
| 327 | M83738 | PTPN9 | protein tyrosine | 0.4 | 70 | 14 |
| | | | phosphatase, non-receptor | | | |
| | | | type 9 | | | |
| 328 | AA210846 | TOM | putative mitochondrial outer | 0.4 | 85 | 17 |
| | | | membrane protein import | | | |
| | | | receptor | | | |
| 329 | AF007892 | P2RY6 | pyrimidinergic receptor P2Y, | 0.4 | 85 | 17 |
| | | | G-protein coupled, 6 | | | |
| 330 | X75593 | RAB13 | RAB13, member RAS | 0.4 | 80 | 16 |
| | | | oncogene family | | | |
| 331 | L26584 | RASGRF1 | Ras protein-specific guanine | 0.4 | 75 | 15 |
| | | | nucleotide-releasing factor 1 | | | |
| 332 | AA040149 | RRN3 | RNA polymerase I | 0.4 | 80 | 16 |
| | | | transcription factor RRN3 | | | |
| 333 | AB012122 | RUVBL1 | RuvB (E coli homolog)-like 1 | 0.4 | 85 | 17 |
| 334 | AA972254 | SACM2L | SAC2 (suppressor of actin | 0.4 | 65 | 13 |
| | | | mutations 2, yeast, | | | |
| | | | homolog)-like | | | |
| 335 | U34044 | SPS | SELENOPHOSPHATE | 0.4 | 90 | 18 |
| | | | SYNTHETASE; Human | | | |
| | | | selenium donor protein | | | |
| 336 | X05403 | SHBG | sex hormone-binding | 0.4 | 85 | 17 |
| | | | globulin | | | |
| 337 | AA366773 | DKFZP566E144 | small fragment nuclease | 0.4 | 70 | 14 |
| 338 | AF044197 | SCYB13 | small inducible cytokine B | 0.4 | 80 | 16 |
| | | | subfamily (Cys-X-Cys motif), | | | |
| | | | member 13 (B-cell | | | |
| | | | chemoattractant) | | | |
| 339 | M24847 | SLC5A1 | solute carrier family 5 | 0.4 | 75 | 15 |
| | | | (sodium/glucose | | | |
| | | | cotransporter), member 1 | | | |
| 340 | J00306 | SST | somatostatin | 0.4 | 75 | 15 |
| 341 | AJ001183 | SOX10 | SRY (sex determining region | 0.4 | 90 | 18 |
| | | | Y)-box 10 | | | |
| 342 | AB006202 | SDHD | succinate dehydrogenase | 0.4 | 75 | 15 |
| | | | complex, subunit D, integral | | | |
| | | | membrane protein | | | |
| 343 | AF013591 | SUDD | sudD (suppressor of bimD6, | 0.4 | 65 | 13 |
| | | | Aspergillus nidulans) | | | |
| | | | homolog | | | |
| 344 | L12350 | THBS2 | thrombospondin 2 | 0.4 | 80 | 16 |
| 345 | U74611 | TNFRSF12 | tumor necrosis factor | 0.4 | 85 | 17 |
| | | | receptor superfamily, | | | |
| | | | member 12 (translocating | | | |
| | | | chain-association membrane | | | |
| | | | protein) | | | |
| 346 | U94788 | TP53 | tumor protein p53 (Li- | 0.4 | 90 | 18 |
| | | | Fraumeni syndrome) | | | |
| 347 | D49677 | U2AF1RS2 | U2 small nuclear | 0.4 | 90 | 18 |
| | | | ribonucleoprotein auxiliary | | | |
| | | | factor, small subunit 2 | | | |
| 348 | AA010724 | LSM2 | U6 snRNA-associated Sm- | 0.4 | 80 | 16 |
| | | | like protein | | | |
| 349 | AI343722 | UQCR | ubiquinol-cytochrome c | 0.4 | 75 | 15 |
| | | | reductase (6.4 kD) subunit | | | |
| 350 | M19720 | MYCL1 | v-myc avian | 0.4 | 80 | 16 |
| | | | myelocytomatosis viral | | | |
| | | | oncogene homolog 1, lung | | | |
| | | | carcinoma derived | | | |
| 351 | X98260 | ZRF1 | zuotin related factor 1 | 0.4 | 65 | 13 |
| 352 | AA610700 | | EST | 0.4 | 85 | 17 |
| 353 | AA678375 | | EST | 0.4 | 80 | 16 |
| 354 | AA393442 | | ESTs | 0.4 | 80 | 16 |
| 355 | R40699 | | ESTs | 0.4 | 90 | 18 |
| 356 | H05961 | | ESTs | 0.4 | 75 | 15 |
| 357 | H89713 | | ESTs | 0.4 | 80 | 16 |
| 358 | R44995 | | ESTs | 0.4 | 75 | 15 |
| 359 | T03765 | | ESTs | 0.4 | 70 | 14 |
| 360 | AI241461 | | ESTs | 0.4 | 75 | 15 |
| 361 | N69098 | | ESTs | 0.4 | 70 | 14 |
| 362 | AA533505 | | ESTs | 0.4 | 70 | 14 |
| 363 | H72347 | | ESTs | 0.4 | 70 | 14 |
| 364 | R64448 | | ESTs | 0.4 | 70 | 14 |
| 365 | AI219995 | | ESTs, Weakly similar to | 0.4 | 60 | 12 |
| | | | Y961_HUMAN | | | |
| | | | HYPOTHETICAL ZINC | | | |
| | | | FINGER PROTEIN | | | |
| | | | KIAA0961 [H. sapiens] | | | |
| 366 | AA885710 | | Homo sapiens cDNA: | 0.4 | 70 | 14 |
| | | | FLJ20886 fis, clone | | | |
| | | | ADKA03257 | | | |
| 367 | AI052376 | | Homo sapiens cDNA: | 0.4 | 80 | 16 |
| | | | FLJ22054 fis, clone | | | |
| | | | HEP09634 | | | |
| 368 | AI332310 | DKFZp762H1311 | hypothetical protein | 0.4 | 60 | 12 |
| | | | DKFZp762H1311 | | | |
| 369 | AI341234 | DKFZP762I166 | hypothetical protein | 0.4 | 85 | 17 |
| | | | DKFZp762I166 | | | |
| 370 | AA286856 | FLJ10466 | hypothetical protein | 0.4 | 70 | 14 |
| | | | FLJ10466 | | | |
| 371 | AA569394 | FLJ12716 | hypothetical protein | 0.4 | 70 | 14 |
| | | | FLJ12716 | | | |
| 372 | AA536091 | FLJ20151 | hypothetical protein | 0.4 | 80 | 16 |
| | | | FLJ20151 | | | |
| 373 | H64396 | FLJ20531 | hypothetical protein | 0.4 | 70 | 14 |
| | | | FLJ20531 | | | |
| 374 | AI290636 | FLJ22215 | hypothetical protein | 0.4 | 60 | 12 |
| | | | FLJ22215 | | | |
| 375 | AA400457 | KIAA0603 | KIAA0603 gene product | 0.4 | 90 | 18 |
| 376 | AI266225 | DKFZP564G202 | DKFZP564G202 protein | 0.4 | 70 | 14 |

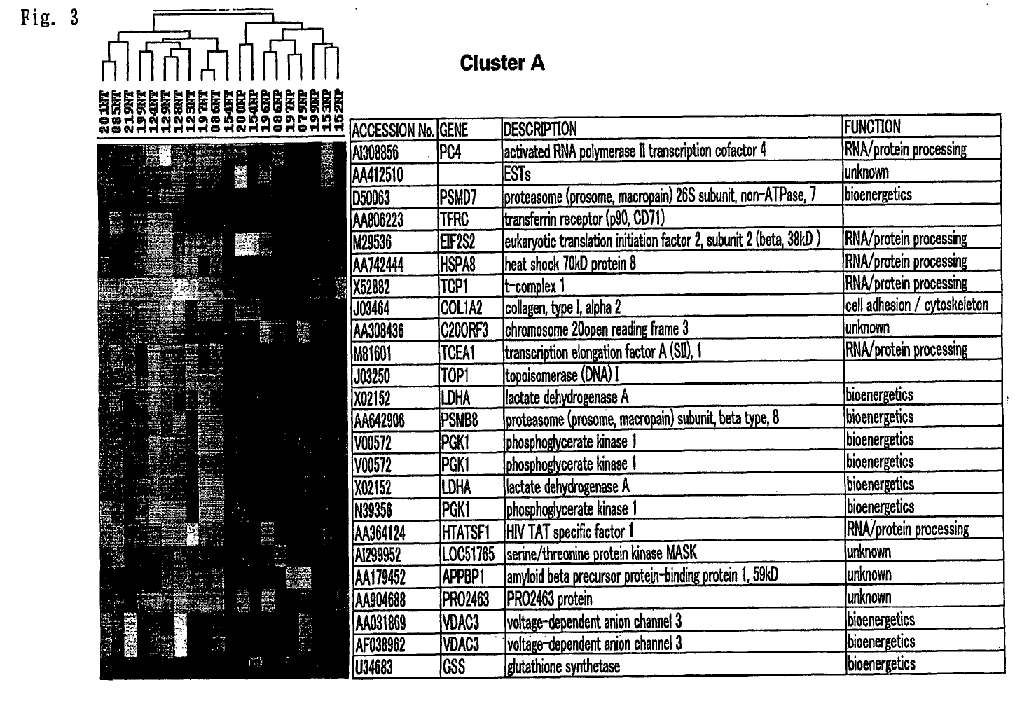

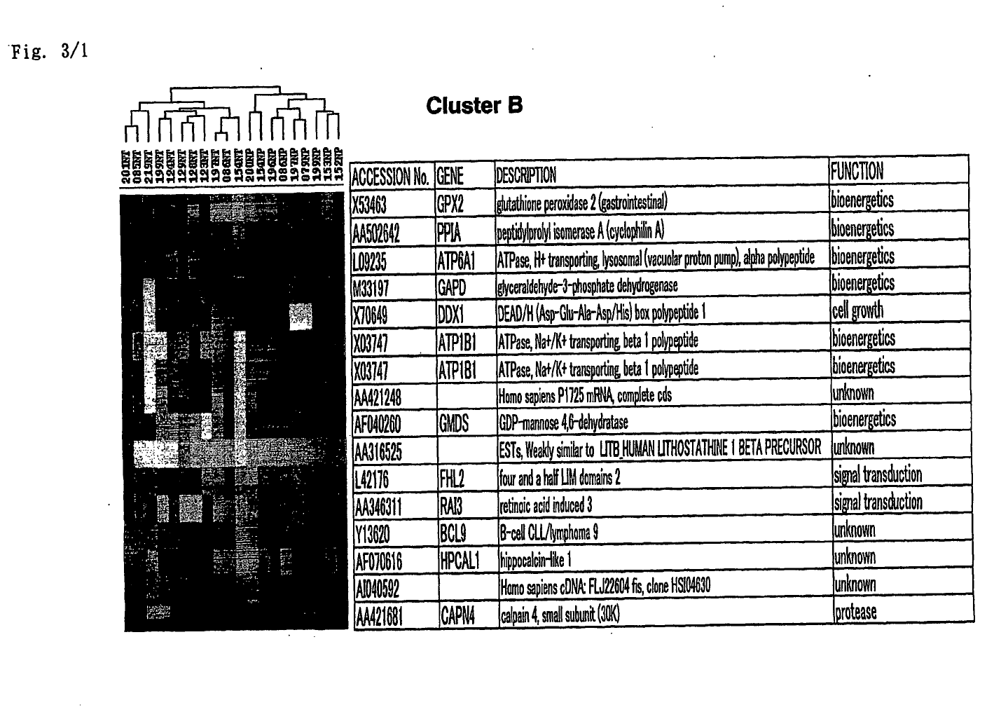

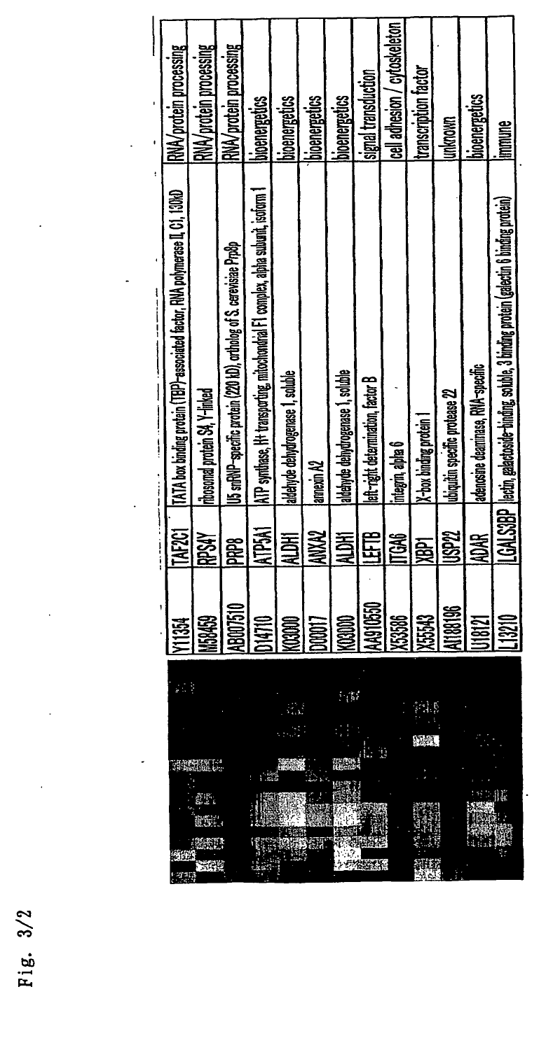

When the present inventors focused on genes in clusters A and B (FIG. 3), they identified several that regulate bioenergetics. In cluster A, PGK1 and LDHA may be induced by hypoxia (Semenza et al., 1994). Proteasomes (PSMD7 and PSMB8) have been reported to accumulate in cancer cells due to glucose starvation and hypoxia (Ogiso et al., 1999). VDAC3 is one of the voltage-dependent anion-selective channel proteins that plays an important role in regulating mitochondrial homeostasis (Vander Heiden et al., 2000). GSS is a regulator of oxidative stress (Uhlig and Wendel, 1992). Some of the genes in cluster B encode proteins that have been reported to function in adaptation to low-oxygen conditions (GPX2, PP1A, GAPD, AXAM2, ALDH1 and ADAR) (Chu et al., 1993; Zhong and Simous, 1999; Hoeren et al., 1998; Denko et al., 2000), in energy consumption (ATP6A1, ATP]B1, and ATPSA1) (Wodopia et al., 2000) or in carbohydrate metabolism (GMDS).

Verification of Microarray Data By Quantitative-RT-PCR

To examine the reliability of the microarray data, six genes were selected for verification. TGFB1 and L4P18 were up-regulated in both adenomas and carcinomas and the others (HECH, NME1, TCEA1 and PSMA7) were differently expressed between adenomas and carcinomas. Their expression was examined in 13 additional paired aRNA samples (7 adenomas and 6 carcinomas) by quantitative RT-PCR (QRT-PCR). The results confirmed the microarray data for all six genes (FIGS. 4A-B). These data verified the reliability and rationale of the strategy to identify genes that are commonly up-regulated or differently expressed during development and progression of colorectal cancer.

Comparison of Expression Analysis Data in Colon Cancers

The data was compared with two sets of data reported previously. First, information of gene expression profiles in two colon cancer tissues and two non-cancerous colonic mucosae analyzed by means of Serial Analysis of Gene Expression was provided by National Center for Biotechnology Information fttp://www.ncbi.nlm.nih.gov/SAGE/). Among the 100 tags of genes expressed most differently between the cancer and non-cancerous tissues, 50 tags corresponded to independent unique genes in UniGene database. Among the 50 genes corresponding to these 50 tags, four up-regulated genes and 24 down-regulated genes in cancer were contained in the microarray. One of the four up-regulated gene, TGFBI (transforming growth factor, beta-induced, 68 kD) also showed elevated expression (FIG. 2). 18 of the 24 down-regulated genes including TSPAN-1 (tetraspan1), GPA33 (glycoprotein A33), CA1 (carbonic anhydrase 1), MT2A (metallothionein 2A), CEACAM1 (carcinoembryonic antigen-related cell adhesion molecule 1), YF13H12 (protein expressed in thyroid), MUC13 (mucin 13), HLAB (major histocompatibility complex class 1B), DUSP1 (dual specificity phosphatase 1), GSN(gelsolin), LGALS4 (galectin 4), CKB (creatinine kinase, brain type), KRT19 (keratin 19), RVASE1 (RNase A family 1), LFI27 (interferon, alpha-inducible protein 27), PP1201, EPS8R2 (FLJ21935), and an EST(Hs.107139) revealed decreased expression in more than half cases examined.

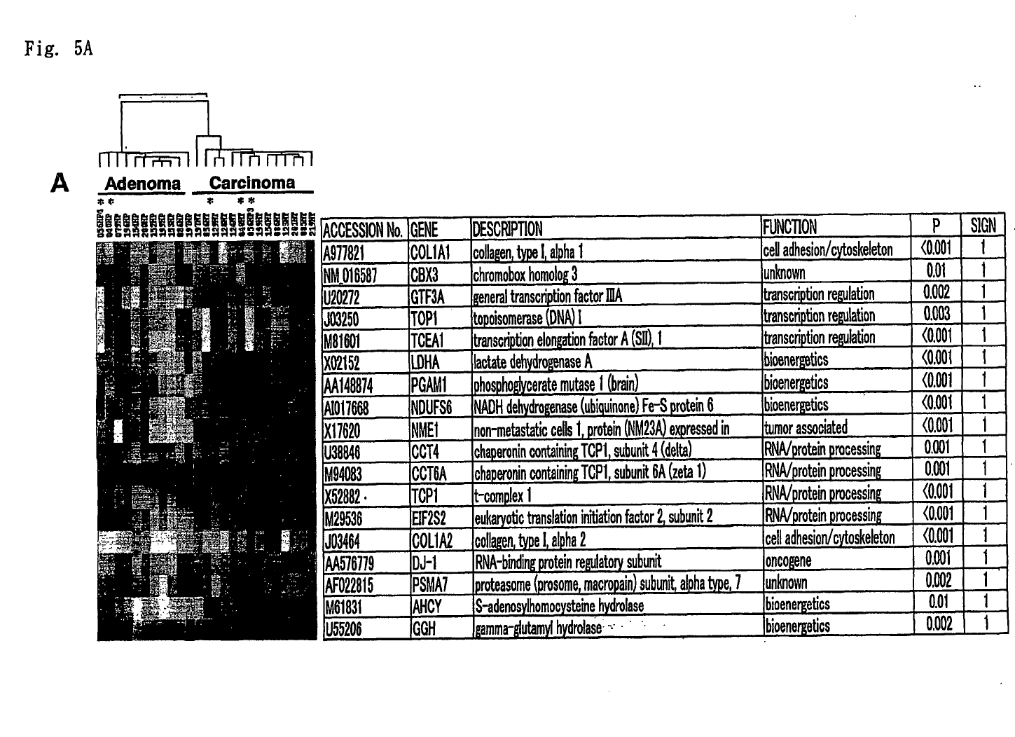

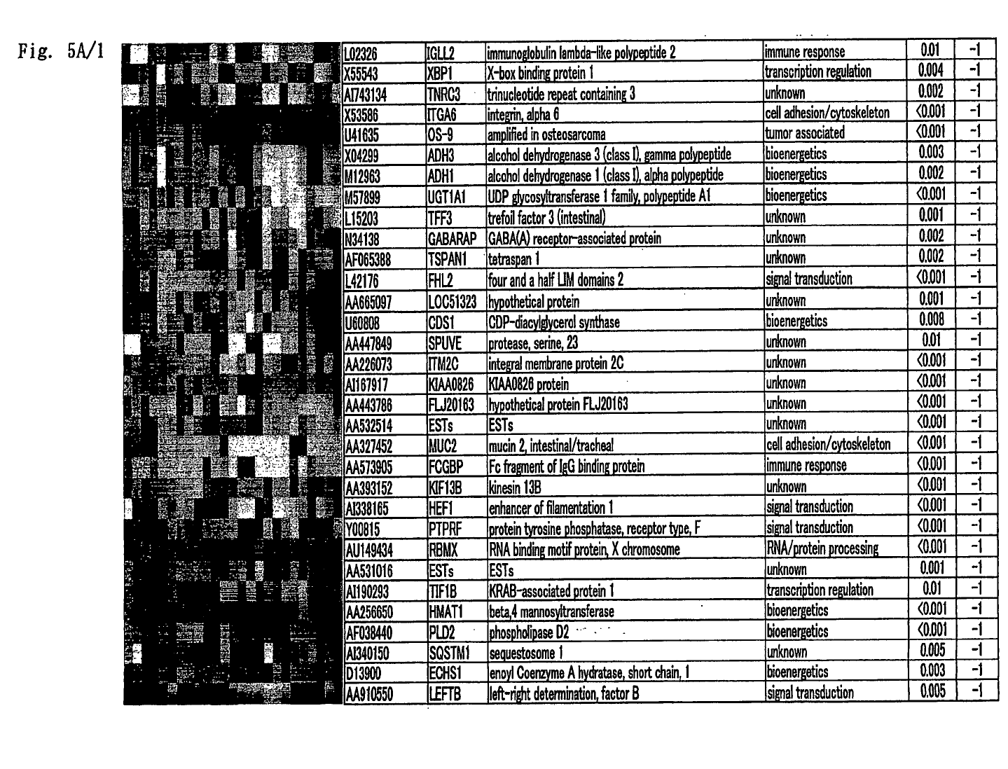

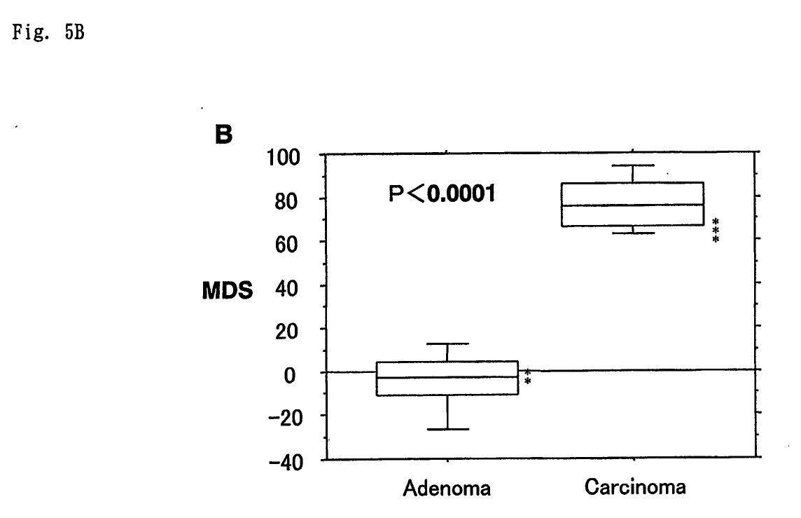

Next, genes highly expressed in colon cancer tissues were compared to matched non-cancerous tissues, which was reported by Notterman et al. using the Affymetrix Human 6500 GeneChip Set Results showed that only two genes, GTF3A (general transcription factor IIIA) and AHCY (adenosylhomocysteine hydrolase), were in the list of frequently up-regulated genes in cancer (FIG. 5A). However, among the ten other genes that were highly expressed in their cancer tissues and spotted on the array slides, eight genes, such as KIAA0101, PYCR1 (pyrroline 5-carboxylate reductase), HSPE1 (heat shock 10 kD protein 1), CDC25B (cell division cycle 25B), CSE1L (chromosome segregation 1-like), CKS2 (CDC28 protein kinase 2), MMP1 (metalloprotenase 1), and CLNS1A (chloride channel, nucleotide-sensitive, 1A), also showed enhanced expression in more than half of cancer tissues.

Development of a “Molecular Diagnosis Score” (MDS) System

Among the genes expressed differently between adenomas and carcinomas, 50 genes whose expression showed statistically significant differences between the two types of tumors were identified (P ≦0.01, Mann-Whitney U test; FIG. 5A). Table 3 shows a list of genes up-regulated in colorectal adenoma as compared to normal tissues, and as can be seen, no significant difference was observed between carcinoma and normal tissues. Table 4 shows a list of genes up-regulated in colorectal carcinoma as compared to normal tissues, and as can be seen, no significant difference was observed between adenoma and normal tissues.

TABLE 3
Marker genes up-regulated in adenoma

| | ACCESSION | | | | |
|---|---|---|---|---|---|
| No. | No. | GENE | DESCRIPTION | P | SIGN |
| 1 | L02326 | IGLL2 | immunoglobulin lambda-like | 0.01 | −1 |
| | | | polypeptide 2 | | |
| 2 | X55543 | XBP1 | X-box binding protein | 0.004 | −1 |
| 3 | AI743134 | TNRC3 | trinucleotide repeart containing 3 | 0.002 | −1 |
| 4 | X53586 | ITGA6 | integrin, alpha 6 | <0.001 | −1 |
| 5 | U41635 | OS-9 | amplified in osteosarcoma | <0.001 | −1 |
| 6 | X04299 | ADH3 | alcohol dehydrogenase 3 (class I), | 0.003 | −1 |
| | | | gamma polypeptide | | |
| 7 | M12963 | ADH1 | alcohol dehydrogenase 3 (class I), | 0.002 | −1 |
| | | | alpha polypeptide | | |
| 8 | M57899 | UGT1A1 | UDP glycosyltransferase 1 family, | <0.001 | −1 |
| | | | polypeptide A1 | | |
| 9 | L15203 | TFF3 | trefoil factor 3 (intestinal) | 0.001 | −1 |
| 10 | N34138 | GABARAP | GABA(A) recepter-associated | 0.002 | −1 |
| | | | protein | | |
| 11 | AF065388 | TSPAN1 | tetraspan 1 | 0.002 | −1 |
| 12 | L42176 | FHL2 | four and a half LIM domeins 2 | <0.001 | −1 |
| 13 | AA665097 | LOC51323 | hypothetical protein | 0.001 | −1 |
| 14 | U60808 | CDS1 | CDP-diacylglycerol synthase | 0.008 | −1 |
| 15 | AA447849 | SPUVE | protease, serine, 23 | 0.01 | −1 |
| 16 | AA226073 | ITM2C | integral membrane protein 2C | <0.001 | −1 |
| 17 | AI167917 | KIAA0826 | KIAA0826 protein | <0.001 | −1 |
| 18 | AA443786 | FLJ20163 | hypothetical protein FLJ20163 | <0.001 | −1 |
| 19 | AA532514 | ESTs | ESTs | <0.001 | −1 |
| 20 | AA327452 | MUC2 | mucin 2, intestinal/tracheal | <0.001 | −1 |
| 21 | AA573905 | FCGBP | Fc fragment of IgG binding protein | <0.001 | −1 |
| 22 | AA393152 | KIF13B | kinesin 13B | <0.001 | −1 |
| 23 | AI338165 | HEF1 | enhancer of filamentation 1 | <0.001 | −1 |
| 24 | Y00815 | PTPRF | protein tyrosine phosphatase, | <0.001 | −1 |
| | | | receptor type, F | | |
| 25 | AU149434 | RBMX | RNA binding motif protein, X | <0.001 | −1 |
| | | | chromosome | | |
| 26 | AA531016 | ESTs | ESTs | 0.001 | −1 |
| 27 | AI190293 | TIF1B | KRAB-associated protein 1 | 0.01 | −1 |
| 28 | AA256650 | HMAT1 | beta, 4 mannosyltransferase | <0.001 | −1 |
| 29 | AF038440 | PLD2 | phospholipase D2 | <0.001 | −1 |
| 30 | AI340150 | SQSTM1 | sequestome 1 | 0.005 | −1 |
| 31 | D13900 | ECHS1 | enoyl Coenzyme A hydratase, | 0.003 | −1 |
| | | | short chain, 1 | | |
| 32 | AA910550 | LEFTB | left-right determination, factor B | 0.005 | −1 |

TABLE 4
Marker genes up-regulated in carcinoma

| No. | ACCESSION No. | GENE | DESCRIPTION | P | SIGN |
|---|---|---|---|---|---|
| 1 | A977821 | COL1A1 | collagen, type 1, alpha 1 | <0.001 | 1 |
| 2 | NM_016587 | CBX3 | chromobox homolog 3 | 0.01 | 1 |
| 3 | U20272 | GTF3A | general transcription facter IIIA | 0.002 | 1 |
| 4 | J03250 | TOP1 | topoisomerase(DNA) 1 | 0.003 | 1 |
| 5 | M81601 | TCEA1 | transcription elongation factor A | <0.001 | 1 |
| | | | (SII), 1 | | |
| 6 | X02152 | LDHA | lactate dehydrogenase A | <0.001 | 1 |
| 7 | AA148874 | PGAM1 | phosphoglycerate mutase 1 | <0.001 | 1 |
| | | | (brain) | | |
| 8 | AI017668 | NDUFS6 | NADH dehydrogenase | <0.001 | 1 |
| | | | (ubiquinone) Fe—S protein 6 | | |
| 9 | X17620 | NME1 | non-metastatic cells 1, protein | <0.001 | 1 |
| | | | (NM23A) expressed in | | |
| 10 | U38846 | CCT4 | chaperonin containing TCP1, | 0.001 | 1 |
| | | | subunit 4 (delta) | | |
| 11 | M94083 | CCT6A | chaperonin containing TCP1, | 0.001 | 1 |
| | | | subunit 6A (zeta 1) | | |
| 12 | X52882 | TCP1 | t-complex 1 | <0.001 | 1 |
| 13 | M29536 | EIF2S2 | eukaryotic translation initiation | <0.001 | 1 |
| | | | factor 2, subunit 2 | | |
| 14 | J03464 | COL1A2 | collagen, type 1, alpha 2 | <0.001 | 1 |
| 15 | AA576779 | DJ-1 | RNA-binding protein regulatory | 0.001 | 1 |
| | | | subunit | | |
| 16 | AF022815 | PSMA7 | proteasome | 0.002 | 1 |
| | | | (prosome, macropain) subunit, | | |
| | | | alpha type 7 | | |
| 17 | M61831 | AHCY | S-adenosylhomocysteine | 0.01 | 1 |
| | | | hydrolase | | |
| 18 | U55206 | GGH | gamma-glutamyl hydrolase | 0.002 | 1 |

Based on expression profiles of these 50 genes, a “Molecular Diagnosis Score” (MDS) system was developed as a way to apply that information to clinical diagnosis. The mean score of the 1 1 carcinomas was 77.4±11.6, while that of the 9 adenomas was −5.9±14.4 (mean±SD, P <0.0001, Mann-Whitney U test; FIG. 5B). The cut-off value for discriminating adenocarcinoma from adenoma was defined as 35, an average of the mean values of the two groups.

Five additional tumors were analyzed to verify the reliability of the MDS system. Among the five samples tested, three that showed scores greater than 35 (73.5, 63.2, and 10 64.6); all turned out to be carcinomas by- histological examination. The two samples with scores of less than 35 (10.3 and -1.4, respectively) were both adenomas (FIG. 5B). Since the distribution of MDSs for 14 carcinomas ranging from 57.7 to 94.1 were completely separated from that for 11 adenomas ranging from −33.4 to 16.7, both sensitivity and specificity of the MDS system were 100% on the basis of this cut-off value. In addition, a hierarchical clustering analysis of all 25 samples correctly separated adenomas from carcinomas based on the expression profiles of the 50 selected genes (FIG. 5A).

Characterization of Colon Cancers By Genome Wide Expression Profiling

Defining characteristics of adenoma and adenocarcinoma of the colon were determined through the analysis of genome-wide expression profiles of patient-derived tissue samples. The 51 genes commonly up-regulated in both adenomas and carcinomas included 19 involved in RNA/protein processing; e.g. ribosomes, translation elongation/initiation factors, and chaperoning. Ribosomes are the molecular machines that manufacture proteins according to blueprints of mRNAs that encode them. Interactions of the ribosome with mRNAs, tRNAs, and a number of non-ribosomal protein cofactors such as translation initiation/elongation factors guarantee that polypeptide chains are initiated, elongated and terminated. After translation, polypeptides emerge from the ribosomes and enter the endoplasmic reticulum where chaperonins may remodel the polypeptides. The data indicate that accelerated protein synthesis appears to be a common feature of adenomas and carcinomas and reflects a heavy proliferative burden in both tumor types. In addition, the data suggest that activation of oncogenes, aberrant transduction of signals, deregulation of the cell cycle, impaired growth control, and remodeling of cytoskeletal structures are general features of tumor cells. The genes (and/or the molecules encoded by the genes) are therapeutic targets for the prevention, diagnosis, and treatment of colorectal cancer.

The down-regulated genes defined herein included a number of genes associated with cell death, which indicates that broad repression of programmed cell-death pathways is a crucial step for colorectal tumorigenesis. In addition, the list of commonly down-regulated genes suggests that reduction of growth-suppressive signals and/or tumor-suppressive functions may confer continuous proliferative properties to neoplastic cells. Increasing the expression of genes categorized in this cluster (e.g., by stimulating expression of the endogenous gene or introducing additional copies of the gene using in vivo or ex vivo gene therapy) may be used to prevent the development of cancer in patients at risk of developing these cancers or treat patients suffering from cancers.

A number of genes which discriminate carcinoma from adenoma were identified and found to be relevant to hypoxia Carcinoma cells are likely to be more exposed to starved and hypoxic conditions, where carbohydrate/oxygen homeostasis is impaired, than are adenoma cells. The data indicate that cancer cells change their expression profiles in response to low-nutrient and hypoxic conditions. Since hypoxia is a prognostic indicator in a number of tumors, targeting the genes in this category allows identification of micro-environmental changes during malignant transformation, and defines prognostic predictors for colon cancer.

Consideration of the nature of the genes described above leads us to conclude that activation of oncogenes, augmentation of proliferation signals, attenuation of anti-proliferative signals, avoidance of self-destruction machinery, alteration of cell structure, and adaptation to microenvironmental changes, are of great importance for the development and progression of normal colonic mucosal cells to adenocarcinomas. These six features suggest that adenoma and carcinoma cells share several genetic characteristics but have unique expression profiles, and the genes identified using the methods described herein represent targets for blocking malignant transformation.

### INDUSTRIAL APPLICABILITY

The gene-expression analysis of colorectal adenomas and carcinomas described herein, obtained through a combination of laser-capture dissection and genome-wide cDNA microarray, has identified specific genes as targets for cancer prevention and therapy. Based on the expression of a subset of these differentially expressed genes, the present invention provides a molecular diagnosis scoring (MDS) system for identifying colorectal tumors. The MDS system of the present invention is a sensitive, reliable and powerful tool that facilitates sensitive, specific and precise diagnosis of such tumors. This system can be specifically utilized in distinguishing adenomas from carcinomas.

The methods described herein are also useful in the identification of additional molecular targets for prevention, diagnosis and treatment of colorectal tumors. The data reported herein add to a comprehensive understanding of colorectal carcinogenesis, facilitate development of novel diagnostic strategies, and provide clues for identification of molecular targets for therapeutic drugs and preventative agents. Such information contributes to a more profound understanding of colorectal tumorigenesis, particularly adenoma-carcinoma progression, and provide indicators for developing novel strategies for diagnosis, treatment, and ultimately prevention of colorectal carcinomas.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

1. A method of diagnosing colorectal tumors in a subject, the method comprising the steps of:
(a) detecting an expression level of one or more marker genes in a specimen collected from a subject to be diagnosed, wherein the one or more marker genes is selected from the group consisting of the genes listed in Table 1 and the genes listed in Table 2; and
(b) comparing the expression level of the one or more marker genes to that of a control, wherein high expression level of a marker gene from Table 1 or a low expression level of a marker gene from Table 2, as compared to control, is indicative of colorectal tumor.

2. The method of claim 1, wherein the expression level of the one or more marker genes is determined by following steps of:
synthesizing aRNA or cDNA of the marker genes from a specimen;
hybridizing the aRNA or cDNA with probes for marker genes; and
detecting the hybridized aRNA or cDNA with the probes quantifying the amount of mRNA thereof.

3. The method of claim 2, wherein the probes are fixed on a DNA array.

4. A method of screening for a therapeutic agent useful in treating or preventing colorectal tumors, said method comprising the steps of:
contacting a candidate compound with a cell expressing one or more marker genes, wherein the one or more marker genes is selected from the group consisting of the genes listed in Table 1, Table 2, Table 3, and Table 4; and
selecting a compound that reduces the expression level of one or more marker genes selected from Table 1, Table 3, and Table 4, as compared to a control, or enhances the expression of one or more marker genes selected from Table 2, as compared to a control.

5. A method of screening for a therapeutic agent usefuil in treating colorectal tumors, said method comprising the steps of:
administering a candidate compound to a test animal;
measuring the expression level of one or more marker genes in a biological sample from the test animal, wherein the one or more marker genes is selected from the group consisting of the genes listed in Table 1, Table 2, Table 3, and Table 4;
selecting a compound that reduces the expression level of one or more marker genes selected from Table 1, Table 3, and Table 4, as compared to a control, or enhances the expression of one or more marker genes selected from Table 2, as compared to a control.

6. A method of screening for a therapeutic agent useful in treating colorectal tumors, said method comprising the steps of:
contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of the genes listed in Table 1, Table 2, Table 3, and Table 4;
measuring the activity of said reporter gene; and
selecting a compound that reduces the expression level of said reporter gene when said marker gene is selected from Table 1, Table 3, and Table 4 or that enhances the expression level of said reporter gene when said marker gene is selected from Table 2, as compared to a control.

7. A method of screening for a therapeutic agent useful in treating colorectal tumors, said method comprising the steps of:
contacting a candidate compound with a protein encoded by a marker gene, wherein the marker gene is selected from the group consisting of the genes listed in Table 1, Table 2, Table 3, and Table 4;
measuring the activity of said protein; and
selecting a compound that reduces the activity of said protein when said marker gene is selected from Table 1, Table 3, and Table 4 or that enhances the activity of said protein when said marker gene is selected from Table 2.

8. The method of any one of claims 4, 5, 6 and 7, wherein the marker gene is selected from the group consisting of the genes listed in Table 3, and wherein colorectal tumor is colorectal adenoma.

9. The method of any one of claims 4, 5, 6 and 7, wherein the marker gene is selected from the group consisting of the genes listed in Table 4, and wherein colorectal tumor is colorectal carcinoma.

10. A method for treating or preventing colorectal tumors, said method comprising the step of administering a compound that is obtained by the method according to any one of claims 4, 5, 6 and 7 .

11. A method for treating or preventing colorectal tumors in a subject, said method comprising the step of administering to the subject an antisense nucleic acids against a marker gene, wherein said marker gene is selected from the group consisting of the genes listed in Table 1, Table 3, and Table 4.

12. A method for treating or preventing colorectal tumors in an subject, said method comprising the step of administering to the subject an antibody or fragment thereof that binds to a protein encoded by a marker gene, wherein said marker gene is selected from the group consisting of the genes listed in Table 1, Table 3, and Table 4.

13. The method of claim 11 or 12, wherein said marker gene is selected from the group consisting of the genes listed in Table 3, and wherein colorectal tumor is colorectal adenoma.

14. The method of claim 11 or 12, wherein said marker gene is selected from the group consisting of the genes listed in Table 4, and wherein colorectal tumor is colorectal carcinoma.

15. A method of treating colorectal tumors in a subject, said method comprising the step of administering to the subject a protein encoded by a marker gene, wherein said marker gene is selected from the group consisting of genes listed in Table 2.

16. A method for vaccinating a subject against colorectal tumors, the method comprising the step of administering:
(a) a DNA corresponding to one or more marker genes selected from the group consisting of the genes listed in Table 1, Table 3, and Table 4.,
(b) a protein encoded by a marker gene, or
(c) an antigenic fragment of a protein encoded by a marker gene either alone, or in combination.

17. The method of claim 16, wherein said marker gene is selected from the group consisting of the genes listed in Table 3, and wherein colorectal tumor is colorectal adenoma.

18. The method of claim 16, wherein said marker gene is selected from the group consisting of the genes listed in Table 4, and wherein colorectal tumor is colorectal carcinoma.

19. A vaccine composition for treating or preventing a colorectal tumor, wherein the vaccine composition comprises one or more components selected from the group consisting of:
(a) DNA corresponding to one or more marker genes selected from the group consisting of the genes listed in Table 1, Table 3, and Table 4.,
(b) a protein encoded by a marker gene, and
(c) an antigenic fragment of a protein encoded by a marker gene.

20. A method of diagnosing adenoma, the method comprising the steps of:
(a) detecting an expression level of one or more marker genes in a specimen collected from a subject to be diagnosed, wherein the one or more marker genes is selected from the group consisting of the genes listed in Table 3; and
(b) comparing the expression level of the one or more marker genes to that of a control, wherein high expression level of a marker gene selected from Table 3, as compared to the control, is indicative of adenoma.

21. A method of diagnosing carcinoma, the method comprising the steps of:
(a) detecting an expression level of one or more marker genes in a specimen collected from a subject to be diagnosed, wherein the one or more marker genes is selected from the group consisting of the genes listed in Table 4; and
(b) comparing the expression level of the one or more marker genes to that of a control, wherein high expression level of a marker gene selected from Table 4, as compared to the control, is indicative of carcinoma.

22. A method of diagnosing adenoma and carcinoma, the method comprising the steps of
(a) detecting an expression level of one or more marker genes in a specimen collected from a subject to be diagnosed, wherein the one or more marker genes is selected from the group consisting of the genes listed in Table 3 and Table 4 ; and
(b) comparing the expression level of the one or more marker genes to that of a control, wherein a high expression level of a marker gene selected from Table 3,
as compared to the control, is indicative of adenoma, and wherein a high expression level of a marker gene selected from Table 4, as compared to the control, is indicative of carcinoma.

23. The method of claim 22, wherein the marker genes comprise all of the marker genes set forth in Table 3 and Table 4.

24. The method of claim 23, wherein step (b) further comprises the steps of determining a function of the log ratios of the expression profiles over the selected genes comprising summing the weighted log ratios of the expression profiles over the selected genes, wherein the weight for each gene is a first value when the average log ratio is higher for carcinomas than for adenomas and a second value when the average log ratio is lower for carcinomas than for adenomas.


SEQUENCE LISTING (21 sequences)

SEQ ID NO: 1 - DNA, 27 bp, Artificial
  artificially synthesized primer sequence
tccctgttgtaatcaagaataagacct

SEQ ID NO: 2 - DNA, 25 bp, Artificial
  artificially synthesized primer sequence
cagttcctttgcagtaagatccatc

SEQ ID NO: 3 - DNA, 36 bp, Artificial
  artificially synthesized probe sequence
ttgttgaaggtctccctattaatgatttctcacgtg

SEQ ID NO: 4 - DNA, 21 bp, Artificial
  artificially synthesized primer sequence
gcagactctgcgcttgagatc

SEQ ID NO: 5 - DNA, 20 bp, Artificial
  artificially synthesized primer sequence
gggctagtcgcacagacctc

SEQ ID NO: 6 - DNA, 26 bp, Artificial
  artificially synthesized probe sequence
aacaagcatcagcgttttccagggct

SEQ ID NO: 7 - DNA, 19 bp, Artificial
  artificially synthesized primer sequence
caaatggctgccaaactgg

SEQ ID NO: 8 - DNA, 22 bp, Artificial
  artificially synthesized primer sequence
ggattctttgttcttccgcact

SEQ ID NO: 9 - DNA, 31 bp, Artificial
  artificially synthesized probe sequence
cgtttgcgagagaaggataagcacattgaag

SEQ ID NO: 10 - DNA, 24 bp, Artificial
  artificially synthesized primer sequence
gacagcagtggagaattgatgttt

SEQ ID NO: 11 - DNA, 26 bp, Artificial
  artificially synthesized primer sequence
acaatttgaggacacttcatatttgc

SEQ ID NO: 12 - DNA, 22 bp, Artificial
  artificially synthesized probe sequence
tgaggcagacttggtgctggcg

SEQ ID NO: 13 - DNA, 24 bp, Artificial
  artificially synthesized primer sequence
gcatacaagttggcaggaacatta

SEQ ID NO: 14 - DNA, 21 bp, Artificial
  artificially synthesized primer sequence
accacaagccgatctccttct

SEQ ID NO: 15 - DNA, 27 bp, Artificial
  artificially synthesized probe sequence
catggcagtgattctgtggagagtgca

SEQ ID NO: 16 - DNA, 21 bp, Artificial
  artificially synthesized primer sequence
agatgcggaaaaacttgacca

SEQ ID NO: 17 - DNA, 20 bp, Artificial
  artificially synthesized primer sequence
agtcagtctgggtcccacca

SEQ ID NO: 18 - DNA, 26 bp, Artificial
  artificially synthesized probe sequence
aagccatcagagagcatcagatggcc

SEQ ID NO: 19 - DNA, 20 bp, Artificial
  artificially synthesized primer sequence
gcagcgttatacgcagagca

SEQ ID NO: 20 - DNA, 19 bp, Artificial
  artificially synthesized primer sequence
acccacgatgagggcagag

SEQ ID NO: 21 - DNA, 18 bp, Artificial
  artificially synthesized probe sequence
tgggcgcaggccgtttgg